(19) United States
(12) Patent Application Publication
(10) Pub. No.: US 2012/0283293 A1
(43) Pub. Date: Nov. 8, 2012

(54) Methods of neuroprotection involving Prostaglandin E2 EP4 (PGE2 EP4) receptor activation

(75) Inventor: Katrin Andreasson, Stanford, CA (US)

(73) Assignee: The Board of Trustees of the Leland Stanford Junior University, Palo Alto, CA (US)

(21) Appl. No.: 13/465,025

(22) Filed: May 6, 2012

Related U.S. Application Data
(60) Provisional application No. 61/483,370, filed on May 6, 2011.

(51) Int. Cl. A61K 31/192; A61P 25/00; A61P 25/28; A61K 31/343; A61K 31/216; A61K 31/45; A61K 31/41; A61P 29/00; A61P 25/16

(52) U.S. Cl. 514/327; 514/570; 514/530; 514/382; 514/468

(57) ABSTRACT

The present invention provides methods for attenuating neuronal inflammation and neuronal damage in case of acute or chronic injury of nerve cells of the central nervous system through prostaglandin E2 EP4 receptor activation. Methods for treating neuropathic pain are also provided.

### RELATED APPLICATION

This application claims priority and other benefits from U.S. Provisional Patent Application Ser. No. 61/483,370, filed May 6, 2011, entitled “Methods of neuroprotection involving prostaglandin E2 EP4 (PGE2 EP4) receptor activation”. Its entire content is specifically incorporated herein by reference.

### STATEMENT OF GOVERNMENT SUPPORT

This invention was made with government support under AG033914 and AG030209 awarded by the National Institutes of Health. The government has certain rights in the invention.

### INCORPORATION OF SEQUENCE LISTING

A computer readable form of the sequence listing, “ASB038 UTL_ST25.txt”, submitted via EFS-WEB, is herein incorporated by reference.

### TECHNICAL FIELD OF THE INVENTION

The present invention relates to methods of attenuating neuroinflammation and neuronal damage involving the administration of agents that activate the prostaglandin E2 EP4 receptor (‘PGE2 EP4 agonists’ or ‘EP4 receptor agonists).

### BACKGROUND

Neuroinflammation resulting from the innate immune responses in the central nervous system to insults such as traumatic brain injury, cerebral ischemia, cerebral glucose deprivation, cerebral oxidative stress, spinal cord injury and excitotoxic injury plays a critical and causative role in the pathogenesis and disease progression of many neurodegenerative diseases as well as acute central nervous system injuries and contributes to aging in the mammalian brain. Attenuating the inflammatory response would be an important step towards reducing the extent of neuronal damage that results from neuroinflammation. It would be highly desirable to have effective neuroprotective methods available to attenuate the inflammatory response that leads to neuroinflammation and ultimately to neuronal damage.

### SUMMARY OF THE INVENTION

Provided herein are methods for attenuating neuronal inflammation and neuronal damage in a human subject following an acute or chronic injury of nerve cells of the central nervous system, comprising the administration of a prostaglandin E2 EP4 receptor agonist in a dosage and dosing regimen effective to attenuate neuronal inflammation and neuronal damage.

Furthermore provided herein are methods for attenuating neuronal inflammation and neuronal damage in a human subject at risk of developing a chronic central nervous system injury, comprising the administration of a prostaglandin E2 EP4 receptor agonist prior to onset of symptoms of a chronic central nervous system injury in a dosage and dosing regimen effective to attenuate neuronal inflammation and neuronal damage.

Furthermore provided herein are methods for treating neuropathic pain that is caused by an inflammatory response in a human subject, comprising the administration of a prostaglandin E2 EP4 receptor agonist in a dosage and dosing regimen effective to treat neuropathic pain, whereby said agonist reduces said inflammatory response by reducing levels of one or more inflammatory cytokines.

In one aspect of the present invention, the prostaglandin E2 EP4 receptor agonist is a protein or a biologically active fragment thereof; a peptide or a biologically active fragment thereof, a peptidomimetic, or a small molecule. In one embodiment of the present invention, the prostaglandin E2 EP4 receptor agonist is AE1-329. In another embodiment, the prostaglandin E2 EP4 receptor agonist is an analog of AE1-329. In a further embodiment, the prostaglandin E2 EP4 receptor agonist is a prodrug of AE1-329. The prostaglandin E2 EP4 receptor agonist can be administered locally at or near a site of injury or systemically.

The above summary is not intended to include all features and aspects of the present invention nor does it imply that the invention must include all features and aspects discussed in this summary.

### INCORPORATION BY REFERENCE

All publications and patent applications mentioned in this specification are herein incorporated by reference to the same extent as if each individual publication or patent application was specifically and individually indicated to be incorporated by reference. In addition, U.S. application Ser. No. 12/763,872, filed on Apr. 20, 2010, and entitled “Treatment of ischemic episodes and cerebroprotection through Prostaglandin E2 (PGE2) EP2 and/or EP4 receptor agonists” as well as Shi et al., 2010, “The Prostaglandin E₂ E-Prostanoid 4 Receptor Exerts Anti-Inflammatory Effects in Brain Innate Immunity”, J Immunol 184:7207-7218 are specifically and entirely incorporated by reference.

### BRIEF DESCRIPTION OF THE DRAWINGS

The accompanying drawings illustrate embodiments of the invention and, together with the description, serve to explain the invention. These drawings are offered by way of illustration and not by way of limitation; it is emphasized that the various features of the drawings may not be to-scale.

FIG. 1 illustrates that EP4 receptor expression is dynamically regulated in BV-2 microglial-like cells, in primary microglia, and in hippocampus in response to lipopolysaccharide (LPS) stimulation, as further detailed in Example 1. (A) Murine BV-2 cells were stimulated with vehicle or LPS (10 ng/ml), and EP4 mRNA was measured at 6 h by qPCR (p<0.001; n=3 per condition). (B) EP4 mRNA is also dynamically regulated in rat primary microglia derived from cerebral cortex and hippocampus (ANOVA p<0.001; by post hoc analysis p<0.05 at 1 h, p<0.001 at 3 h, and p<0.05 at 6 h; n=3 per condition). (C) EP4 mRNA is upregulated at 6 h in mouse hippocampus and returns to baseline by 24 h after peripheral administration of LPS (5 mg/kg IP; n=3-6 per group; p<0.05). (D) Confocal 400× imaging is shown of microglial cells in the hilar region of hippocampus from vehicle-treated and LPS-treated mice (5 mg/kg IP at 6 hours after stimulation). EP4 signal is localized in Iba1 positive microglia (arrows) in a punctate perinuclear distibution in both vehicle and LPS treated mice (scale bar=10 microns).

FIG. 1E illustrates microglial morphology 6 hours after stimulation with vehicle, LPS, or LPS+EP4 agonist, as further detailed in Example 1. 400× representative confocal images are shown of hippocampal CA1 microglia stained with microglial markers Iba1 and CD68 6 hours after administration of vehicle, LPS, or LPS+AE1-329. Iba 1 labels the cytoskeleton, and detects cell soma and processes; CD68 labeling is punctate, as it is localized to endosomes and lysozomes, and is detected prominently in microglial processes. LPS stimulation results in increased Iba1 staining with some reduction in the number of Iba-1 positive ramified processes. Co-administration of EP4 agonist modestly reduces Iba1 staining as compared to LPS alone. Scale bar=25 μm.

FIG. 2 illustrates that EP4 signaling suppresses pro-inflammatory gene transcription in BV-2 cells and primary microglia stimulated with LPS, as further detailed in Example 2. BV-2 cells (A-D) and cerebral cortical microglia (E) were stimulated with LPS (10 ng/ml) or PBS+/− the EP4 agonist AE1-329 (1 μM) or vehicle. (A) qPCR of COX-2, iNOS, and gp91^{phox} in BV-2 cells at 6 h shows a significant increase with LPS treatment in vehicle (v) treated groups (#p<0.001) but a significant decrease with co-administration of AE1-329 (AE; *p<0.05, **p<0.01; n=3 per condition). (B) Expression in BV-2 cells of pro-inflammatory cytokines TNF-α, IL1β, and IL-6 is significantly induced with LPS (#p<0.001) but decreased with co-stimulation of EP4 receptor agonist (*p<0.05). (C) The anti-inflammatory cytokine IL-10 is upregulated with EP4 receptor stimulation (p<0.05). (D) LPS-induced increase in nitrite concentration in BV-2 cells is decreased in a dose-dependent manner with AE1-329 (0-1 μM) at 24 hours (# p<0.001 vehicle vs LPS alone; dose response for AE1-329 ANOVA p<0.0001; post hoc analysis p<0.001 for 0.001, 0.01, 0.1, and 1 μM, n=5 per condition). (E) Primary microglia were stimulated +/−LPS+/−EP4 agonist AE1-329 (100 nM) and harvested at 3 hours. qPCR demonstrates a reduced level of iNOS as well as significant reductions of COX-2, TNF-α, and gp91^{phox} and upregulation of IL-10 in LPS-treated microglia with EP4 receptor agonist (#p<0.01-0.001 for vehicle vs LPS; *p<0.05, ***p<0.001 for LPS vs LPS+AE1-329; n=6 per condition).

FIG. 3 illustrates that EP4 receptor activation in BV-2 cells increases PKA activity and reduces LPS-induced phosphorylation of Akt, as further detailed in Example 2. (A) PKA activity assay of BV-2 cells stimulated with LPS (100 ng/ml), AE1-329 (100 nM), or both shows significant increases with AE1-329 and LPS+AE1-329 (*p<0.05; n=5 samples per condition). (B) Inhibition of PKA with H89 at 5 μM and 10 μM reverses AE1-329-mediated increase in PKA activity (*p<0.05 and **p<0.01). (C) Representative quantitative Western analysis of p-Akt and total Akt shows an increase in p-Akt with LPS (100 ng/ml) treatment that is reduced with stimulation with EP4 agonist AE1-329 (100 nM). BV-2 cells were treated with LPS+/−AE1-329 or vehicle and harvested at time points of 5, 15, 30, and 60 minutes; cell lysates were immunoblotted for phosphorylated Ser473 Akt (p-Akt) and total Akt. The average densitometry from three experiments is shown in the lower panel. p-Akt/Akt values have been normalized to the average signal at time=0 minutes of LPS and LPS+AE1 values. There was a significant effect of AE1-329 treatment [F(1,4)=4.589, p<0.05] and of time [F(1,4)=7.72, p<0.001]. Densitometric measurements of effects of vehicle vs AE1-329 alone did not show differences (data not shown). (D) ELISA of phospho-Thr308 Akt and total Akt at 60 minutes after stimulation with LPS+/−AE1-329 shows a significant increase in p-Akt/Akt levels with LPS stimulation, which is reversed with co-administration of 100 nM AE1-329 (*p<0.05; **p<0.01; n=6 per condition).

FIG. 4 illustrates that EP4 receptor activation in BV-2 cells reduces phosphorylation of IKK and nuclear translocation of NF-κB subunits p65 and p50, as further detailed in Example 3. (A) Representative quantitative Western analysis of phospho-IKK (p-IKK) and total IKK and densitometric average of three experiments demonstrates an increase in phospho-IKK with LPS stimulation (100 ng/ml) that is significantly attenuated with co-activation of the EP4 receptor (AE1, 100 nM; [F(1,4)=4.709, p<0.05] for effect of AE1-329). Densitometric measurements are represented as ratios of p-IKK/IKK and are normalized to time=0 minutes for LPS and LPS+AE1. (B and C) Representative quantitative Western analyses are shown for NF-κB p65 (B) and NF-κB p50. (C) nuclear translocation and cytoplasmic levels in BV-2 cells treated with LPS+/−AE1-329. NF-κB subunit signals were normalized to the nuclear marker lamin B1. Densitometry measurements for nuclear levels of p65 and p50 represent averages of three experiments in which values for individual time points were normalized to the 15 minute vehicle time point. There was a significant effect of AE1-329 treatment for both p65 ([F(1,4)=11.13, p<0.01]) and p50 ([F(κ1,4)=11.88, p<0.01]) nuclear translocation; there was a significant effect of time for both p65 and p50 [F(1,4)=42.7, p<0.001] and [F(1,4)=27.06, p<0.001], respectively. Maximal attenuation of LPS-dependent nuclear translocation is evident by 60 minutes for NF-κB p65 (**p<0.01) and by 120 minutes for p50 (***p<0.001) with activation of the EP4 receptor. (D) Nuclear translocation of NF-κB p65 was quantified in BV-2 cells 60 minutes after stimulation with either LPS (100 ng/ml) or PBS+/−AE1-329 (100 nM) or vehicle. Cells were immunostained for NF-κB p65 and nuclei were counterstained with Hoechst and examined at 400× with confocal microscopy (scale bar=8 microns). Immunofluorescent staining of p65 in control, AE1-329, and LPS+AE1-329 nuclei (top row in red) demonstrates more diffuse and lighter staining (white horizontal arrows), in contrast to the dense nuclear staining in LPS alone (white vertical arrow). (E) Quantification of immunofluorescent nuclear signal intensity of p65 was carried out in BV-2 cells treated with veh, AE1-329, LPS, and LPS+AE1-329. Five fields per condition were measured, representing >100 cells per field. There was a significant increase in nuclear levels of p65 at one hour following LPS stimulation as compared to vehicle alone (***p<0.001), and this increase was significantly attenuated with co-stimulation of the EP4 receptor (**p<0.01).

FIG. 5 illustrates that Cd11bCre conditional deletion of EP4 results in increased pro-inflammatory gene expression and increased lipid peroxidation in brain, as further detailed in Example 4. Hippocampal mRNA and protein were isolated from Cd11bCre:EP4+/+ and Cd11bCre:EP4f/f male mice 24 h after peripheral stimulation with LPS (5 mg/kg i.p.). (A) In Cd11bCre:EP4f/f mice qPCR demonstrates increased expression of COX-2, TNF-α, IL-6, IL-1β, and NADPH oxidase subunits p47^{phox}, p67^{phox}, gp91^{phox}, and iNOS 24 hours after peripheral LPS stimulation (* p<0.05; **p<0.01; n=4-7 male mice per group). (B) Representative quantitative Western analyses and densitometry of p47^{phox} and p67^{phox} in LPS treated Cd11bCre:EP4+/+ versus Cd11bCre:EP4f/f mice (n=4-5 per genotype, *p<0.05, **p<0.01). There were no differences between genotypes treated with vehicle (data not shown). (C) Gas chromatography mass spectrophotometric (GCMS) quantification of lipid peroxidation in cerebral cortical lysates demonstrates a significant increase in F2-isoprostanes (isoPs) in Cd11b: EP4f/f mice 24 h after LPS as compared to control Cd11bCre:EP4+/+mice treated with LPS (n=4-7 per genotype; *p<0.05).

FIG. 5D illustrates that microglial morphology 24 hours after LPS in Cd11bCre:EP4+/+ and Cd11bCre:EP4f/f hippocampus does not show significant differences, as further detailed in Example 4. 630× representative confocal images of hippocampal CA1 microglia are shown 24 hours after treatment with vehicle and LPS in Cd11bCre and Cd11bCre:EP4 f/f mice. Morphological assessment of microglia shows a modest increase in Iba1 immunoreactivity and thickening of processes in LPS versus vehicle treated Cd11bCre mice. Although at this time point of 24 hours there is significant upregulation of pro-inflammatory gene expression and increased lipid peroxidation, there is no clear difference in overall morphology between Cd11bCre and Cd11bCre:EP4 f/f mice. Scale bar=25 μm.

FIG. 6 illustrates that systemic administration of EP4 agonist decreases LPS-induced hippocampal pro-inflammatory gene response, as further detailed in Example 5. Mice were pretreated with AE1-329 (300 μg/kg, s.c.) for 30 min before injection of LPS (5 mg/kg, i.p.) and hippocampal RNA was isolated at 6 hours after LPS. (A) Pro-inflammatory COX-2 and iNOS are strongly induced 6 h after systemic LPS administration, while administration of the selective EP4 agonist AE1-329 blunts induction. (B) Induction of cytokines TNF-α, IL-6, and IL-1β are also decreased with administration of AE1-329 (n=7-8 per group of 3 month male C57B6 mice; # p<0.001 vehicle vs LPS; *p<0.05 LPS/veh vs LPS/AE1-329).

FIG. 7 illustrates that the EP4 receptor regulates inflammatory gene expression in microglia isolated from adult mouse brain, as further detailed in Example 6. Microglia were isolated by density gradient centrifugation from 2-3 mo C57B7 male mice administered saline or LPS+/−EP4 agonist (AE1-329 0.3 mg/kg) or vehicle (A), and from 2-3 mo Cd11bCre:EP4+/+ and Cd11bCre:EP4f/f mice 6 hours and 24 hours after LPS (B). (A) Significant increases in microglial expression of COX-2, iNOS, IL-6, TNF-α, and gp91^{phox} were observed in wild type mice in response to LPS, but these increases were significantly blunted with co-treatment with EP4 agonist (#p<0.01; (*p<0.05; **p<0.01; n=6-8 mice per group). (B) Proinflammatory gene expression is elevated in Cd11bCre:EP4+/+ and Cd11bCre:EP4f/f microglia at 6 hours after LPS; however, increased gene expression persists at 24 h in microglia isolated from Cd11bCre:EP4f/f mice as compared to Cd11bCre:EP4+/+control mice. Gene expression does not return to basal levels for COX-2, IL-1β, and TNF-α at 24 h in Cd11bCre:EP4f/f microglia, and is significantly increased beyond the 6 hour level for iNOS at this late time point (*p<0.05; **p<0.01; n=4-8 per group).

FIG. 8 illustrates that peripheral administration of EP4 reduces LPS-mediated inflammatory response in plasma, as further detailed in Example 7. Plasma was collected and analyzed at 3 hours after co-administration of PBS or LPS (5 mg/kg, i.p.)+/−vehicle or AE1-329 (300 μg/kg, s.c.). (A) Cluster analysis of regulated cytokines and myeloperoxidase (MPO) following peripheral PBS or LPS administration+/−AE1-329. (B) Absolute concentrations of regulated cytokines (pg/ml) and MPO (ng/ml) are decreased with AE1-329 administration.

FIG. 9 shows results from density centrifugation with +/−Cd11b immunomagnetic purification yields >90% Cd11b-expressing microglial cells, as further detailed in Example 7. (A) Flow cytometry of cells from density centrifugation yields 90.39% Cd11b-positive microglia, consistent with published data (de Haas et al., 2007). (B) Immunomagnetic purification yields 97.62% Cd11b-positive microglia.

FIG. 10 illustrates that EP4 receptor activation reduces the expression of the canonical pro-inflammatory cytokines IL-6 and TNF-α in primary human monocytes stimulated with lipopolysaccharide (LPS), as further detailed in Example 8. Primary human monocytes were stimulated with either vehicle (PBS) alone, AE1-329 (100 nM), LPS (100 ng/ml), and LPS (100 ng/ml) plus AE1-329 (100 nM). A) ELISA of TNF-α using anti-human TNF-α at 6 hours after stimulation with LPS shows a significant increase in TNF-α levels (measured in pg/ml, p<0.0001), which was entirely reversed with co-administration of 100 nM of EP4 agonist AE1-329 (p<0.0001). B) ELISA of IL-6 using anti-human IL-6 at 6 hours after stimulation with LPS shows a significant increase in IL-6 levels (measured in pg/ml, p<0.0001), which was significantly reduced with co-administration of 100 nM of EP4 agonist AE1-329 (p<0.0003).

### DETAILED DESCRIPTION

Before describing detailed embodiments of the invention, it will be useful to set forth definitions that are utilized in describing the present invention.

### 9.1. Definitions

The practice of the present invention may employ conventional techniques of chemistry, molecular biology, recombinant DNA, microbiology, cell biology, immunology and biochemistry, which are within the capabilities of a person of ordinary skill in the art. Such techniques are fully explained in the literature. For definitions, terms of art and standard methods known in the art, see, for example, Sambrook and Russell ‘Molecular Cloning: A Laboratory Manual’, Cold Spring Harbor Laboratory Press (2001); ‘Current Protocols in Molecular Biology’, John Wiley & Sons (2007); William Paul ‘Fundamental Immunology’, Lippincott Williams & Wilkins (1999); M. J. Gait ‘Oligonucleotide Synthesis: A Practical Approach’, Oxford University Press (1984); R. Ian Freshney “Culture of Animal Cells: A Manual of Basic Technique’, Wiley-Liss (2000); ‘Current Protocols in Microbiology’, John Wiley & Sons (2007); ‘Current Protocols in Cell Biology’, John Wiley & Sons (2007); Wilson & Walker ‘Principles and Techniques of Practical Biochemistry’, Cambridge University Press (2000); Roe, Crabtree, & Kahn ‘DNA Isolation and Sequencing: Essential Techniques’, John Wiley & Sons (1996); D. Lilley & Dahlberg ‘Methods of Enzymology: DNA Structure Part A: Synthesis and Physical Analysis of DNA Methods in Enzymology’, Academic Press (1992); Harlow & Lane ‘Using Antibodies: A Laboratory Manual: Portable Protocol No. I’, Cold Spring Harbor Laboratory Press (1999); Harlow & Lane ‘Antibodies: A Laboratory Manual’, Cold Spring Harbor Laboratory Press (1988); Roskams & Rodgers ‘Lab Ref: A Handbook of Recipes, Reagents, and Other Reference Tools for Use at the Bench’, Cold Spring Harbor Laboratory Press (2002). Each of these general texts is herein incorporated by reference.

Unless defined otherwise, all technical and scientific terms used herein have the same meaning as commonly understood by a person of ordinary skill in the art to which this invention belongs. The following definitions are intended to also include their various grammatical forms, where applicable. As used herein, the singular forms “a” and “the” include plural referents, unless the context clearly dictates otherwise.

The term “prostaglandin E2 EP4 receptor agonist”, as used herein, relates to biologically active, recombinant, isolated peptides and proteins, including their biologically active fragments, peptidomimetics and small molecules that are capable of stimulating the prostaglandin E2 EP4 receptor.

The terms “prostaglandin E2 EP4 receptor agonist”, “PGE2 EP4 receptor agonist” and “EP4 receptor agonist”, “prostaglandin E2 EP4 agonist”, “PGE2 EP4 agonist” and “EP4 agonist” are used interchangeably herein.

The term “pharmaceutical composition”, as used herein, refers to a mixture of a PGE2 EP4 receptor agonist with chemical components such as diluents or carriers that do not cause unacceptable, i.e. counterproductive to the desired therapeutic effect, adverse side effects and that do not prevent the PGE2 EP4 receptor agonist from exerting a therapeutic effect. A pharmaceutical composition serves to facilitate the administration of the PGE2 EP4 receptor agonist.

The term “therapeutic effect”, as used herein, refers to a consequence of treatment that might intend either to bring remedy to an injury that already occurred or to prevent an injury before it occurs. A therapeutic effect may include, directly or indirectly, the reduction of neuronal inflammation (neuroinflammation) and neuronal damage following acute or chronic injury of nerve cells. A therapeutic effect may also include, directly or indirectly, the arrest, reduction, or elimination of the progression of neuronal cell death following acute or chronic injury of nerve cells. Furthermore, a therapeutic effect may include, directly or indirectly, the reduction of neuronal inflammation and neuronal damage prior to the onset of symptoms of a chronic injury of nerve cells. Symptoms of a chronic injury of nerve cells in a human subject may be recognized by repeatedly assessing the cognitive function of the human subject over the course of a particular time period, for instance, over the course of several weeks, months or years. Furthermore, a therapeutic effect may include, directly or indirectly, the attenuation, alleviation, reduction and elimination of neuropathic pain that is caused by an inflammatory response through the release of one or more cytokines such as TNF-α, IL-1β and IL-6.

The terms “therapeutically effective amount” and “dosage effective to attenuate neuronal inflammation and neuronal damage” of a PGE2 EP4 receptor agonist relate to an amount of a PGE2 EP4 receptor agonist that is sufficient to provide a desired therapeutic effect in a human subject. Naturally, dosage levels of the particular PGE2 EP4 receptor agonist employed to provide a therapeutically effective amount vary in dependence of the type of injury, the age, the weight, the gender, the medical condition of the human subject, the severity of the condition, the route of administration, and the particular PGE2 EP4 receptor agonist employed. Therapeutically effective amounts of a PGE2 EP4 receptor agonist, as described herein, can be estimated initially from cell culture and animal models. For example, IC₅₀ values determined in cell culture methods can serve as a starting point in animal models, while IC₅₀ values determined in animal models can be used to find a therapeutically effective dose in humans.

The term “dosing regimen”, as used herein, refers to the administration schedule and administration intervals of the particular prostaglandin E2 EP4 receptor agonist employed to obtain the desired therapeutic effect.

The term “analog of AE1-329” refers to compounds and molecules that are similar in chemical structure (“structural analog”) to AE1-329, which is a small molecule that is also known as ONO-AE1-329, but that can be different with respect to functional groups, number of carbon atoms, substructure or substitution. A compound or molecule that is similar in functional activity (“functional analog) to AE1-329 can also be an analog of AE1-329. The systematic chemical name of AE1-329 according to the International Union of Pure and Applied Chemistry (IUPAC) is 2-[3-[(1R,2S,3R)-3-hydroxy-2-RE,3S)-3-hydroxy-5-[2-(methoxymethyl)phenyl]pent-1-enyl]-5-oxocyclopentyl]sulfanylpropylsulfanyl]acetic acid. Examples of a structural analog of AE1-329 is ONO-4819 with the systematic chemical name of methyl 7-[(1R,2R,3R)-3-hydroxy-2-[(E)-(3S)-3-hydroxy-4-(m-methoxymethylphenyl)-1-butenyl]-5-oxocyclopentyl]-5-thiaheptanoate (Yoshida et al., 2002) or AGN205203 with the systematic chemical name of 7-[2-(3-Hydroxy-4-phenyl-but-1-enyl)-6-oxo-piperidin-1-yl]-heptanoic acid methyl ester, C₂₃H₃₃NO₄) (Jiang et al., 2007). Further examples of structural analogs are APS-999 with the systematic chemical name of (1R,2R,3aS,8bS)-5-(2-(1H-tetrazol-5-yl)ethyl)-1-((S,E)-4-cyclohexyl-3-hydroxybut-1-enyl)-2,3,3a,8b-tetrahydro-1H-cyclopenta[b][1]benzofuran-2-ol and APS-856 with the systematic, chemical name of 3-((1R,2R,3aS,8bS)-1-((S,E)-4-cyclohexyl-3-hydroxybut-1-enyl)-2-hydroxy-2,3,3a,8b-tetrahydro-1H-cyclopenta[b][1]benzofuran-5-propanoic acid (Hayashi et al., 2011)

Examples of functional analogs to AE1-329 are compounds or molecules that activate the prostaglandin E2 EP4 receptor, but that have a different chemical structure. The degree in the difference in chemical structure can be pronounced or can be subtle. Often the difference in the chemical structure refers to the backbone structure of the compound or molecule, which usually is a N-heterocyclic structure.

The term “prodrug” refers to a compound or molecule that is administered as a functionally inactive form of AE1-329 or of an AE1-329 analog and requires bioactivation in the body of a human subject to become functionally active. Prodrugs of a functional compound are often produced as esters of the functional compound. In this example, bioactivation, i.e. metabolization into an functionally active metabolite, happens by means of hydrolysis of the prodrug to the functional compound.

The term “recombinant”, as used herein, relates to a protein or polypeptide that is obtained by expression of a recombinant polynucleotide.

The terms “isolated” and “purified”, as used herein, relate to molecules that have been manipulated to exist in a higher concentration or purer form than naturally occurring.

The terms “neuronal inflammation” and “neuroinflammation” are used interchangeably herein.

The term “neuronal damage”, as used herein, relates to various forms of cognitive impairment. Cognitive impairment can reduce the capacity of individuals to learn, remember, communicate, socialize, problem solve, and/or function independently. It may be due to a neurodegenerative disorder caused by genetic and/or environmental factors, or it may be an acquired condition. Neuronal damage, leading to acutely injured or degenerating neurons, can also result from aberrant, excessive stimulation of neurons through excitatory neurotransmitters (excitotoxicity), such as the excitatory neurotransmitter glutamate.

### 9.2. Dosages, Dosing Regimens, Formulations and Administration of Prostaglandin E2 Ep4 Receptor Agonists

The dosage and dosing regimen for the administration of a prostaglandin E2 EP4 receptor agonist for attenuating neuroinflammation and neuronal damage or for treating neuropathic pain, as provided herein, is selected by one of ordinary skill in the art, in view of a variety of factors including, but not limited to, age, weight, gender, and medical condition of the subject, the severity of the inflammatory response that is experienced, the route of administration (oral, systemic, local), the dosage form employed, and may be determined empirically using testing protocols, that are known in the art, or by extrapolation from in vivo or in vitro tests or diagnostic data.

The dosage and dosing regimen for the administration of a prostaglandin E2 EP4 receptor agonist, as provided herein, is also influenced by toxicity in relation to therapeutic efficacy. Toxicity and therapeutic efficacy can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Molecules that exhibit large therapeutic indices are generally preferred.

The therapeutically effective dose of a prostaglandin E2 EP4 receptor agonist, can, for example, be less than 50 mg/kg of subject body mass, less than 40 mg/kg, less than 30 mg/kg, less than 20 mg/kg, less than 10 mg/kg, less than 5 mg/kg, less than 3 mg/kg, less than 1 mg/kg, less than 0.3 mg/kg, less than 0.1 mg/kg, less than 0.05 mg/kg, less than 0.025 mg/kg, or less than 0.01 mg/kg. Therapeutically effective doses of a prostaglandin E2 EP4 receptor agonist, administered to a subject as provided in the methods herein can, for example, can be between about 0.001 mg/kg to about 50 mg/kg. In certain embodiments, the therapeutically effective dose is in the range of, for example, 0.005 mg/kg to 10 mg/kg, from 0.01 mg/kg to 2 mg/kg, or from 0.05 mg/kg to 0.5 mg/kg. In various embodiments, an effective dose is less than 1 g, less than 500 mg, less than 250 mg, less than 100 mg, less than 50 mg, less than 25 mg, less than 10 mg, less than 5 mg, less than 1 mg, less than 0.5 mg, or less than 0.25 mg per dose, which dose may be administered once, twice, three times, or four or more times per day. In certain embodiments, an effective dose can be in the range of, for example, from 0.1 mg to 1.25 g, from 1 mg to 250 mg, or from 2.5 mg to 1000 mg per dose. The daily dose can be in the range of, for example, from 0.5 mg to 5 g, from 1 mg to 1 g, or from 3 mg to 300 mg.

In some embodiments, the dosing regimen is maintained for at least one day, at least two days, at least about one week, at least about two weeks, at least about three weeks, at least about one month, three months, six months, one year, three years, six years or longer. In some embodiments, an intermittent dosing regimen is used, i.e., once a month, once every other week, once every other day, once per week, twice per week, and the like.

Routes of administration of prostaglandin E2 EP4 receptor agonists or pharmaceutical compositions containing prostaglandin E2 EP4 receptor agonists may include, but are not limited to, oral, nasal and topical administration and intramuscular, subcutaneous, intravenous, intraperitoneal or intracerebral injections. The prostaglandin E2 EP4 receptor agonists or pharmaceutical compositions containing prostaglandin E2 EP4 receptor agonists may also be administered locally into the central nervous system via an injection or in a targeted delivery system.

The prostaglandin E2 EP4 receptor agonist may be administered in a single daily dose, or the total daily dose may be administered in divided doses, two, three, or more times per day. Optionally, in order to reach a steady-state concentration in the brain quickly, an intravenous bolus injection of the prostaglandin E2 EP4 receptor agonist can be administered followed by an intravenous infusion of the prostaglandin E2 EP4 receptor agonist.

The prostaglandin E2 EP4 receptor agonist can be administered to the subject as a pharmaceutical composition that includes a therapeutically effective amount of the prostaglandin E2 EP4 receptor agonist in a pharmaceutically acceptable vehicle. It can be incorporated into a variety of formulations for therapeutic administration by combination with appropriate pharmaceutically acceptable carriers or diluents, and may be formulated into preparations in solid, semi-solid, liquid, or gaseous forms, such as tablets, capsules, powders, granules, ointments, solutions, suppositories, injections, inhalants, gels, microspheres, and aerosols.

In some embodiments, the prostaglandin E2 EP4 receptor agonist can be formulated as a delayed release formulation. Suitable pharmaceutical excipients and unit dose architecture for delayed release formulations may include those described in U.S. Pat. Nos. 3,062,720 and 3,247,066. In other embodiments, the prostaglandin E2 EP4 receptor agonist can be formulated as a sustained release formulation. Suitable pharmaceutical excipients and unit dose architecture for sustained release formulations include those described in U.S. Pat. Nos. 3,062,720 and 3,247,066. The prostaglandin E2 EP4 receptor agonist can be combined with a polymer such as polylactic-glycoloic acid (PLGA), poly-(I)-lactic-glycolic-tartaric acid (P(I)LGT) (WO 01/12233), polyglycolic acid (U.S. Pat. No. 3,773,919), polylactic acid (U.S. Pat. No. 4,767,628), poly(ε-caprolactone) and poly(alkylene oxide) (U.S. 20030068384) to create a sustained release formulation. Such formulations can be used in implants that release an agent over a period of several hours, a day, a few days, a few weeks, or several months depending on the polymer, the particle size of the polymer, and the size of the implant (see, e.g., U.S. Pat. No. 6,620,422). Other sustained release formulations are described in EP 0 467 389 A2, WO 93/241150, U.S. Pat. No. 5,612,052, WO 97/40085, WO 03/075887, WO 01/01964A2, U.S. Pat. No. 5,922,356, WO 94/155587, WO 02/074247A2, WO 98/25642, U.S. Pat. Nos. 5,968,895, 6,180,608, U.S. 20030171296, U.S. 20020176841, U.S. Pat. Nos. 5,672,659, 5,893,985, 5,134,122, 5,192,741, 5,192,741, 4,668,506, 4,713,244, 5,445,832 4,931,279, 5,980,945, WO 02/058672, WO 9726015, WO 97/04744, and. US20020019446. In such sustained release formulations microparticles of drug are combined with microparticles of polymer. Additional sustained release formulations are described in WO 02/38129, EP 326 151, U.S. Pat. No. 5,236,704, WO 02/30398, WO 98/13029; U.S. 20030064105, U.S. 20030138488A1, U.S. 20030216307A1,U.S. Pat. No. 6,667,060, WO 01/49249, WO 01/49311, WO 01/49249, WO 01/49311, and U.S. Pat. No. 5,877,224.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients, and the like. The compositions can also include additional substances to approximate physiological conditions, such as pH adjusting and buffering agents, toxicity adjusting agents, wetting agents, and detergents. The composition can also include any of a variety of stabilizing agents, such as an antioxidant for example. Tablet formulations can comprise a sweetening agent, a flavoring agent, a coloring agent, a preservative, or some combination of these to provide a pharmaceutically elegant and palatable preparation.

Further guidance regarding formulations that are suitable for various types of administration can be found in Remington's Pharmaceutical Sciences, Mace Publishing Company, Philadelphia, Pa., 20th ed. (2000).

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in-vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Prostaglandin E2 EP4 receptor agonists or pharmaceutical compositions containing prostaglandin E2 EP4 receptor agonists may be administered to a subject using any convenient means capable of resulting in the desired attenuation of neuroinflammation and neuronal damage or treatment of neuropathic pain. Routes of administration include, but are not limited to, oral, rectal, parenteral, intravenous, intracranial, intraperitoneal, intradermal, transdermal, intrathecal, intranasal, intracheal, intracapillary, subcutaneous, subdermal, topical, intramuscular, injection into the cerebrospinal fluid, injection into the intracavity, or injection directly into the brain. Oral administration can include, for instance, buccal, lingual, or sublingual administration. The prostaglandin E2 EP4 receptor agonists may be systemic after administration or may be localized by the use of local administration, intramural administration, or use of an implant that acts to retain the active dose at the site of implantation. For a brief review of methods for drug delivery see Langer, 1990.

### 9.3. The Innate Immune Response, Neuronal Inflammation and Neuronal Damage

Neuronal inflammation is a powerful, utterly destructive force in the progressive nature of neurodegenerative diseases and also compromises neuronal viability in cases of acute injury and the general aging process. Significant insight into the critical role of neuronal inflammation in neurodegenerative disease has been gained from studies of the innate immune response because of considerable overlap in cellular and molecular inflammatory mechanisms (Nguyen et al., 2002; Letiembre et al., 2007a/b). Innate immune response with its immediate (minutes to hours) reaction after an infectious challenge plays a significantly larger role in the pathogenesis of neurodegenerative diseases than adaptive immune response does, since the latter requires several days to proliferate T and B lymphocytes in response to a specific pathogen or antigen and, so, to become effective.

A well-studied animal model of innate immunity in brain involves the (systemic) administration of the bacterial endotoxin lipopolysaccharide (LPS). The administration of LPS, also referred to herein as LPS stimulation or stimulation with LPS, induces the expression of IL-1α and IL-1β, tumor necrosis factor alpha (TNF-α), IL-6 and other pro-inflammatory cytokine mRNAs and proteins in the brain. The peripheral immune response to LPS can be transmitted to brain parenchyma in several ways: by direct effects on circumventricular organs or perivascular macrophages, stimulation of vagal afferents, direct transport of cytokines into brain, and transduction of serum immune responses to parenchyma via endothelial cells. The resulting CNS innate immune response is characterized by activation of microglial cells and generation of neurotoxic reactive oxygen species, cytokines, and proteases that lead to neuronal and synaptic injury and behavioral deficits (Qin et al., 2007; McGeer & McGeer, 2004; Liu et al., 2008).

Aging is also associated with an increased activity of the innate immune system and consequently an enhanced production of pro-inflammatory cytokines, such as IL-6, in the brain, while the production of anti-inflammatory cytokines, such as IL-10, is decreased, thereby disturbing the natural balance between pro- and anti-inflammatory cytokines.

### The Innate Immune Response

Glial cells are non-neuronal cells that surround and insulate neurons from one another, supply oxygen and nutrients to neurons, destroy pathogens and remove dead neurons. Microglia are the smallest of the glial cells and are generally considered the resident innate immune cells of the brain and the spinal cord, particularly because of their phagocytic activity, acting as the first form of active immune defense in the central nervous system. A wide variety of viral, fungal, bacterial and protozoal components are able of evoking an innate immune response. Microglial cells can become activated by a single stimulus such as lipopolysaccharide, lipopeptides, yeast wall mannans, bacterial DNA (Abreu & Arditi, 2004), and in response release neurotoxic factors, including tumor necrosis factor-α, nitric oxide, interleukin-1α, interleukin-1β, and reactive oxygen species, all causing neuronal damage. Chronic microglial activation and repeated release of such proinflammatory neurotoxic factors drive progressive neuron damage in cases of neurodegenerative diseases (Lull & Block, 2010; Letiembre 2007a).

Recently, toll-like receptors (TLRs) have been identified as key players in the protective mechanism of the innate immune response; all toll-like receptors share a common activation pathway resulting in the nuclear translocation and activation of the proinflammatory transcription factor NF-κB. The first described mammalian toll-like receptor, TLR4, is responsible for the recognition of the bacterial lipopolysaccharide (LPS, endotoxin), which is found in the outer membrane of various gram-negative bacteria and can cause septic shock. In humans, LPS binds to the serum lipid binding protein and is then transferred to the pattern recognition receptor CD14, then to the MD-2 protein which associates with TLR4.

Neuronal cell death as a consequence of apoptotic or necrotic events can be caused in acute and chronic ways through neuronal damage and neuronal inflammation. Acute neuronal injury and acute neurodegeneration can be caused by a traumatic brain injury due to a sudden, violent insult, by cerebral ischemia due to restricted blood supply, glucose deprivation, oxidative stress through free radicals or spinal cord injury. Neurodegenerative diseases of the central nervous system (CNS) such as Alzheimer's disease, Parkinson's disease, Amyotrophic Lateral Sclerosis and Huntington's disease lead to chronic neurodegeneration and often manifest themselves in cognitive impairment. The risk of developing a neurodegenerative disorder generally increases with age.

Excitotoxic injury (excitatory amino acid neurotoxic injury) following overstimulation of the glutamate receptors, the NMDA receptor or the AMPA receptor, by the neurotransmitter glutamate or molecules with a similar effect (so-called excitotoxins), such as N-methyl D-aspartate (NMDA) or kainic acid, may be involved in both acute and chronic neurodegenerative events.

### 9.3. Neuropathic Pain and the Inflammatory Response

Neuropathic pain is characterized by a hypersensitivity to chemical or mechanical stimuli as well as a sensation of pain to harmless stimuli. Neuropathic pain is a pathological type of chronic pain that persists even when the pain-evoking stimuli is removed and the resulting damage is resolved; its negative impact on the quality of a human subject's life is significant (McDermott et al., 2006). The most common types of neuropathic pain are post-herpetic neuralgia, trigeminal neuralgia and diabetic neuropathy (Leung and Cahill, 2010). Proinflammatory cytokines such as IL-1α and IL-1β, IL-6, and particularly tumor necrosis factor-alpha (TNF-α), besides other proinflammatory cytokines, and also chemokines have been recognized as instrumental in the pathogenesis of neuropathic pain (Leung and Cahill, 2010). PGE₂ EP4 receptor activation mediates an anti-inflammatory effect in the central nervous system by blocking the expression of proinflammatory cytokines and offers thus a new treatment approach for neuropathic pain.

### 9.4. The Prostaglandin E2 EP4 Receptor

The prostaglandin E2 EP4 receptor is a member of the G-protein coupled receptor family and is encoded by the PTGER4 gene in humans. Prostanoids including various prostaglandins (PGs) and thromboxanes (TXs) are cyclooxygenase (COX) metabolites of C₂₀-unsaturated fatty acids such as arachidonic acid, whereby the cyclooxygenases COX-1 and COX-2 catalyze the first committed step in the synthesis. Prostanoids exert a variety of actions in various tissues and cells. The most typical actions are the relaxation and contraction of various types of smooth muscles. They also modulate neuronal activity by either inhibiting or stimulating neurotransmitter release, sensitizing sensory fibers to noxious stimuli, or inducing central actions such as fever generation and sleep induction. Among prostanoids, the E type prostaglandins are most widely produced in the body and exhibit the most versatile actions through four different G-protein-coupled receptors designated EP₁, EP₂, EP₃, and EP₄, resulting in changes in the production of cAMP and/or phosphoinositol turnover, intracellular Ca²⁺ mobilization and agonist-induced changes in activities of downstream kinases (Coleman et al., 1994; Narumiya et al., 1999).

The PGE2 EP4 receptor is positively coupled to cAMP and its expression is strongly induced in brain upon systemic LPS administration (Zhang & Rivest, 1999).

### 9.5. Prostaglandin E2 EP4 Receptor Agonists

The present invention provides methods for attenuating neuronal inflammation and neuronal damage as well as methods for treating neuropathic pain using agents that stimulate the prostaglandin E2 EP4 receptor in case of an acute or chronic injury of nerve cells of the central nervous system to counteract the resulting inflammatory response. Prostaglandin E2 EP4 receptor agonists may be biologically active, recombinant, isolated peptides and proteins, including their biologically active fragments, peptidomimetics or small molecules. In the working examples the small molecule AE1-329, with the systematic chemical name of 243-[(1R,2S ,3R)-3-hydroxy-2-[(E,3S)-3-hydroxy-5-[2-(methoxymethyl)phenyl]pent-1-enyl]-5-oxocyclopentyl]sulfanylpropylsulfanyl]acetic acid, was utilized as prostaglandin E2 EP4 receptor agonist to stimulate and activate the PGE2 EP4 receptor.

Prostaglandin E2 EP4 receptor agonists can be identified experimentally using a variety of in vitro and/or in vivo models. Isolated prostaglandin E2 EP4 receptor agonists can be screened for binding to various sites of the purified prostaglandin E2 EP4 receptor proteins. Compounds can also be functionally screened for their ability to exert anti-inflammatory effects using in vitro culture systems as well as in vivo animal models (e.g., monkey, rat, or mouse models). Candidate compounds that exert anti-inflammatory effects may also be identified by known pharmacology, structure analysis, or rational drug design using computer based modeling.

Candidate compounds that exert anti-inflammatory effects may encompass numerous chemical classes, though typically they are organic molecules, preferably small organic compounds having a molecular weight of more than 50 and less than about 2,500 daltons. They may comprise functional groups necessary for structural interaction with proteins (e.g., hydrogen bonding), and typically include at least an amine, carbonyl, hydroxyl, or carboxyl group. They often comprise cyclical carbon or heterocyclic structures and/or aromatic or polyaromatic structures substituted with one or more functional groups. They may be found among biomolecules including peptides, saccharides, fatty acids, steroids, purines, and pyrimidines, and structural analogs thereof.

Candidate compounds that exert anti-inflammatory effects can also be synthesized or isolated from natural sources (e.g., bacterial, fungal, plant, or animal extracts). The synthesized or isolated candidate compound may be further chemically modified (e.g., acylated, alkylated, esterified, or amidified), or substituents may be added (e.g., aliphatic, alicyclic, aromatic, cyclic, substituted hydrocarbon, halo (especially chloro and fluoro), alkoxy, mercapto, alkylmercapto, nitro, nitroso, sulfoxy, sulfur, oxygen, nitrogen, pyridyl, furanyl, thiophenyl, or imidazolyl substituents) to produce structural analogs, or libraries of structural analogs (see, for example, U.S. Pat. Nos. 5,958,792; 5,807,683; 6,004,617; 6,077,954). Such modification can be random or based on rational design (see, for example, Cho et al., 1998; Sun et al., 1998).

Such prostaglandin E2 EP4 receptor agonists may be administered locally at or near a site of injury within the central nervous system or systemically in a dosage and dosage regimen that is effective to provide the desired therapeutic effect.

### 9.6. Neuroprotective Utility of Prostaglandin E2 Ep4 Receptor (PGE2 Ep4) Agonists: Attenuating Neuroinflammation and Neuronal Damage; Treating Neuropathic Pain

As described in Example 1, PGE2 EP4 receptor expression is upregulated in microglial-like cells as well as in primary microglia cells in response to lipopolysaccharide stimulation. Upon activation of the PGE2 EP4 receptor with a selective agonist, pro-inflammatory gene transcription is suppressed, as described in Example 2.

As described in Example 5, PGE₂ EP4 receptor activation mediates an anti-inflammatory effect in brain by blocking LPS-induced pro-inflammatory gene expression in mice which was associated in cultured murine microglial cells with decreased Akt and IKK phosphorylation and decreased nuclear translocation of p65 and p50 NF-kappaB subunits, as further detailed in Examples 2 and 3.

As described in Example 4, conditional deletion of the PGE2 EP4 receptor in macrophages and microglia increased lipid peroxidation and pro-inflammatory gene expression in brain and in isolated adult microglia following peripheral LPS administration. As explained in Example 6, EP4 selective agonist decreased LPS-induced pro-inflammatory gene expression in hippocampus and in isolated adult microglia. In plasma, EP4 agonist significantly reduced levels of pro-inflammatory cytokines and chemokines, indicating that peripheral EP4 activation protects the brain from systemic inflammation (Example 7). In Example 8, EP4 receptor activation attenuated lipopolysaccharide-induced release of proinflammatory cytokines in primary human monocytes.

### 9.7. Assessing Neuroinflammation

As discussed in Example 8, human EP4 receptor activation in LPS-stimulated human monocytes had a distinct anti-inflammatory effect and significantly decreased levels of IL-6 and TNF-α. The degree of neuroinflammation and the degree of attenuating neuroinflammation following administration of PGE2 EP4 receptor agonists in a human subject can, thus, for instance, be assessed by measuring levels of proinflammatory cytokines, such as IL-1α, IL-1β, IL-6 and TNF-α, prior and after administration of the PGE2 EP4 receptor agonist.

### 9.8. Assessing Neuronal Damage/Cognitive Function

As described earlier, neuronal damage can be related to various forms of cognitive impairment. Cognitive impairment can reduce the capacity of individuals to learn, remember, communicate, socialize, problem solve, and/or function independently.

Numerous tests or protocols for assessing cognitive function are known in the art. Such tests can, for instance, be employed to assess a cognitive function or an improvement of a cognitive function through the attenuation of neuronal damage, in a human subject administered with a compound that inhibits the inflammatory response, e.g. a PGE2 EP4 receptor agonist in accordance with the methods provided herein.

The improvement in cognitive function and attenuation of neuronal damage can be determined by measuring a cognitive function in a subject or a population of subjects before and after administration of the dosing regimen of a PGE2 EP4 receptor agonist. In some embodiments, the improvement in cognitive function or the lack in the improvement in cognitive function and lack of attenuation of neuronal damage is determined by measuring a cognitive function in a subject or a population of subjects to whom a PGE2 EP4 receptor agonist, as provided herein, is administered as compared to measurements made in a subject or a population of subjects to whom a PGE2 EP4 receptor agonist is not administered.

Assessing improvement in cognitive function and attenuation of neuronal damage, or the lack thereof, can be evaluated using any test or protocol known in the art. For instance, the Clinician's Global Impression of Change (CGI/C) counts among one of the most commonly used tests to assess overall change in clinical trials. The validity of this type of measure is based on the ability of an experienced clinician to detect a clinically relevant change in a patient's overall clinical state against a trivial change.

Cognitive function in humans can be assessed using any of a number of tests known in the art, including but not limited to tests of IQ, recognition, comprehension, reasoning, remembering, creation of imagery, capacity for judgment, learning and so forth. Assessment tests include, for example, the Diagnostic Adaptive Behavior Scale (DABS), the Wechsler Adult Intelligence Scale (WAIS) including it revisions, the W AIS-R and W AIS-III, the Mini-Mental State Examination (MMSE) or “Folstein” test, the Blessed Information-Memory-Concentration Test (BIMC), the Fuld Object Memory Evaluation (FOME), the California Verbal Learning Test (CVLT) and revised version (CVLT-II), the DAME battery, and so forth.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. In the following, experimental procedures and examples will be described to illustrate parts of the invention.

### EXPERIMENTAL PROCEDURES

The following methods and materials were used in the examples that are described further below.

Materials.

Lipopolysaccharide (LPS, Escherichia coli 055: B5; Calbiochem, La Jolla, Calif.) was resuspended in sterile phosphate-buffered saline (PBS) at 1 mg/ml and stored at −20° C. EP4 specific agonist AE1-329 {16-(3-methoxymethyl) phenyl-omega-tetranor-3,7-dithia prostaglandin E1} was a generous gift from Ono Pharmaceuticals Co., Osaka, Japan. Its selectivity for the EP4 receptor has been previously established (Suzawa et al., 2000; Shibuya et al., 2002). H-89 was purchased from Biomol (Plymouth Meeting, Pa.). Cell culture media, supplements, and antibiotics were purchased from Invitrogen (Carlsbad, Calif.).

Animals.

The murine in vivo studies were conducted in accordance with the National Institutes of Health guidelines for the use of experimental animals and protocols were approved by the Institutional Animal Care and Use Committee. C57B6 EP4 floxed mice (Schneider et al., 2004) were kindly provided by Drs. Richard and Matthew Breyer (Vanderbilt University School of Medicine, Nashville, Tenn.), and C57B6 Cd11bCre mice (Boillee et al., 2006) were kindly provided by Dr. G. Kollias (Alexander Fleming Biomedical Sciences Research Center, Vari, Greece) and Dr. Donald Cleveland (UCSD, San Diego, Calif.). All mice were housed in an environment controlled for lighting (12 hour light/dark cycle), temperature, and humidity, with food and water available ad libitum. Cd11bCre:EP4f/f and Cd11bCre:EP4+/+ mice were generated by serial crosses of C57B6 Cd11bCre and EP4f/f and EP4+/+ lines. Male Cd11bCre: EP4f/f and Cd11bCre: EP4+/+ mice were treated with either saline or LPS (5 mg/kg intraperitoneally: n=5-8 per group, 13 months of age). 24 hours after injection, mice were euthanized and brain tissue was harvested and frozen at −80° C. For pharmacological experiments, C57B6 male mice (Jackson Laboratories, Bar Harbor, Me.; n=7 or 8 per group) received an injection of saline or LPS (5 mg/kg, i.p.+/−vehicle or AE1-329 (300 pg/kg subcutaneously) (Qin et al., 2007; Nagamatsu et al., 2006). Mice were euthanized 6 hours later, and brain tissue was harvested and frozen at −80° C. For collection of plasma, C57B6 male mice (n=5 per group) received an injection of saline or LPS (5 mg/kg, i.p.)+/−AE1-329 (300 pg/kg s.c.) or vehicle. Mice were deeply anesthetized with isoflurane at 3 h and blood was collected in a 1-ml syringe pre-coated with EDTA (250 mM) and placed in EDTA coated tubes. Plasma was collected after centrifugation at 1000×g for 10 min at 4° C. and frozen at −80° C.

Murine Cell Culture.

Murine microglial-like BV-2 cells were grown in DMEM medium supplemented with 10% heat-inactivated fetal bovine serum (HyClone, Logan, Utah) and 100 units/ml each of penicillin and streptomycin and were maintained at 37° C. in a humidified atmosphere containing 5% CO₂. For primary microglial cultures, cerebral cortices were isolated from postnatal day 2 Sprague-Dawley rat pups obtained from Charles River Laboratories International, Inc. (Davis, Calif.). Tissues were minced and incubated in 0.25% trypsin-EDTA, mechanically triturated in DMEM/F-12 with 10% FBS, and plated on poly-L-lysine-coated 75 ml flasks. Cultures were maintained for 14 days with media changes every 4 days. Microglial cells were isolated by shaking flasks at 200 rpm in a Lab-Line™ Incubator-Shaker for 6 h. The purity of microglial cultures was confirmed with immunostaining for Iba1 and was >95% pure. BV-2 cells were seeded onto 6-well or 24-well plates and allowed to grow to 80-90% confluence. Primary microglia were seeded onto 24 well plates at 5×10⁵ cells per ml.

Human Cell Culture.

Human monocyte derived macrophages were purchased from AllCells (Catalog #PB-MDM-001F: Frozen normal peripheral blood monocyte derived macrophages) and plated at a density of 23,000 cells per well in a 96 well plate. Two dilutions (1:5 and 1:10) were tested using anti-human IL6 and anti-human TNFα ELISA to measure concentrations of these two cytokines in pg/ml after 6 hours of stimulation with either vehicle alone, AE1-329 (100 nM), LPS, and LPS plus AE1-329 (100 nM). Activation of the human EP4 receptor significantly reduced levels of IL-6 and TNFα, consistent with the data in mice.

Quantitative Real-Time PCR (qPCR).

qPCR was carried out as previously described (Liang et al., 2008). Briefly, total RNA was isolated using Trizol reagent (Invitrogen, Carlsbad, Calif.), treated with DNAse (Invitrogen), and the reaction was terminated by heating at 65° C. for 10 minutes. First strand cDNA synthesis was performed with 1.5 μg of total RNA, 4 units of Omniscript enzyme (Qiagen, Valencia, Calif.) and 0.25 μg of random primer in a reaction volume of 20 μl at 37° C. for 1 hour. Reverse transcribed cDNA was diluted 1:20 in RNAse free ddH₂O for subsequent RT-PCR. The mRNA level for each target gene was quantified by SYBR Green-based qPCR using the QuantiTect SYBR Green PCR kit (Qiagen). Melting curve analysis confirmed the specificity of each reaction. Forward and reverse oligonucleotide primers for interleukin-6 (IL-6), interleukin-1β (IL-1β), tumor necrosis factor-α (TNF-α), inducible nitric oxide synthase (iNOS), COX-2, NADPH subunits gp91^{phox}, p67^{phox}, p47^{phox}, and interleukin-10 (IL-10) (IDT Integrated DNA Technologies, Coralville, Iowa) are listed in Table 1. The reaction was performed using 50 of cDNA, 0.25-0.5 μM of primer, and 2×SYBR Green Super Mix (Qiagen) with a final volume of 25 μL. Quantification was performed using the standard curve method. Gene expression level was normalized to 18S RNA, and relative mRNA expression is presented relative to control. Samples without reverse transcriptase served as the negative control. PCR assays were performed using the PTC-200 Real Time PCR System (MJ Research). Experiments were repeated in triplicate.

TABLE 1
qRT PCR primers

| | | | Accession |
|---|---|---|---|
| | Sense | Anti-sense | Number |
| 18S | 5′-CGGCTACCACATCCAAGGAA- | 5′-GCTGGAATTACCGCGGCT-3′ | AY248756 |
| | 3′ | | |
| EP4 | 5′- | 5′- | NM_008965 |
| | AGACACCACCTCGCTGAGAACT | AACCTCATCCACCAACAGGACA | |
| | TT-3′ | CT-3′ | |
| p67^{phox} | 5′- | 5′- | NM_010877 |
| | GCCGGAGACGCCAGAAGAGCT | GGGGCTGCGACTGAGGGTGAA- | |
| | A-3′ | 3′ | |
| gP91^{phox} | 5′- | 5′- | NM_007807 |
| | CCAACTGGGATAACGAGTTCA- | GAGAGTTTCAGCCAAGGCTTC-3 | |
| | 3′ | | |
| p47^{phox} | 5′- | 5′- | NM_010876 |
| | TACAGCAAAGGACAGGACTGG | GAGGCACTTGGCTTTCTGCAAA | |
| | GTT-3′ | CT-3′ | |
| iNOS | 5′- | 5′-GCCATCGGGCATCTGGTA-3′ | MMU43428 |
| | TGACGGCAAACATGACTTCAG- | | |
| | 3′ | | |
| COX-2 | 5′- | 5′-GCTCAGTTGAACGCCTTTTG- | NM_011198 |
| | TGCAAGATCCACAGCCTACC-3′ | 3′ | |
| IL-10 | 5′- | 5′- | NM_010548 |
| | GGGTTGCCAAGCCTTATCGGAA | TCTTCAGCTTCTCACCCAGGGAA | |
| | AT-3′ | T-3′ | |
| TNFα | 5′- | 5′- | NM_013693 |
| | GATCTCAAAGACAACCAACATG | CTCCAGCTGGAAGACTCCTCCC | |
| | TG-3′ | AG-3′ | |
| IL-1β | 5′- | 5′- | NM_008361 |
| | CCAGGATGAGGACATGAGCAC | TTCTCTGCAGACTCAAACTCCAC- | |
| | C-3′ | 3′ | |
| IL-6 | 5′- | 5′- | NM_031168 |
| | CATAGCTACCTGGAGTACATGA | CATTCATATTGTCAGTTCTTCG- | |
| | -3′ | 3′ | |

Immunostaining.

Free-floating 40 μm coronal brain sections through hippocampus were generated and processed for immunostaining as previously described (Liang et al., 2005). The following primary antibodies were used: anti-EP4 (1/1000; Cayman Chemicals, Ann Arbor, Mich.) and anti-Iba I (1/500; Wako, Richmond, Va.). Secondary antibodies and detection reagents included donkey anti-mouse Alexa 555, anti-rabbit Alexa 486, and Zenon 555 for detection of Iba1 (Molecular Probes, Eugene, Oreg.). Specific staining of the EP4 antibody was confirmed using blocking peptide and no primary antibody in control experiments. Images were acquired by sequential scanning using the Leica TCS SPE confocal system and DM 5500 Q microscope (Leica Microsystems, Mannheim, Germany) with laser lines 405, 488 and 532 nm. Sections corresponding to 6 μM were selected and equally processed in Leica LAS AF (Leica Microsystems) and collapsed stacks were obtained with MetaMorph software (Molecular Devices, Sunnyvale, Calif.).

Nuclear Extract Preparation.

Nuclear and cytoplasmic fractions of BV-2 cells were prepared at several time points after treatment (0-120 minutes) using the nuclear extract kit from Active Motif (Carlsbad, Calif.). Briefly, cells were washed, collected in ice-cold PBS in the presence of phosphatase inhibitors, and centrifuged at 300×g for 5 min at 4° C. Cell pellets were resuspended in hypotonic buffer, treated with detergent, and centrifuged at 14,000×g for 30 sec at 4° C. After collection of the cytoplasmic fraction, nuclei were solubilized for 30 min in lysis buffer containing protease inhibitors. Lysates were centrifuged at 14,000×g for 30 min at 4° C. and supernatants were collected for NF-κB studies. To prepare whole cell lysates for phospho-Akt and phospho-IKK studies, cells were washed in ice-cold PBS and lysed in 20 mM Tris-HCl, pH 7.5, 150 mM NaCl, 1 mM Na₂EDTA, 1 mM EGTA, 1% Triton, 2.5 mM sodium pyrophosphate, 1 mM β-glycerophosphate, 1 mM orthovanadate, 1 μg/ml leupeptin and 1 mM PMSF. Lysates were sonicated for 5 seconds, centrifuged at 14,000×g for 10 min at 4° C. and supernatants were collected for phospho-Akt and phospho-IKK studies. All protein concentrations were determined using the Bradford protein assay.

Western blot analysis. Protein (20 μg per lane) was fractionated using 12% SDS-PAGE and electrophoretically transferred to PVDF membranes (Bio-Rad, Hercules, Calif.). For phospho-Akt/Akt and phospho-IKK/IKK studies, membranes were probed with anti phospho-Ser473 Akt antibody or anti-phospho-IKK antibody (1:1000, Cell signaling, Beverly, Mass.) and anti-Akt and anti-IKK antibodies (1:1000; Cell Signaling). For NF-κB nuclear translocation studies, membranes were probed with anti-NF-κB p105/p50 antibody (1:5000, Abcom, Cambridge, Mass.) or anti-NF-κB p65 antibody (1:300, Santa Cruz Biotechnology). Loading controls included anti-actin antibody (1:10,000, Santa Cruz Biotechnology, Inc. Santa Cruz, Calif.) for cytosolic fractions and anti-lamin B1 antibody (1:10,000, Abcom, Cambridge, Mass.) for nuclear fractions. Immunoreactivity was detected using either sheep anti-rabbit or sheep anti-mouse HRP-conjugated secondary antibody (Amersham Biosciences, Arlington Heights, Ill.), followed by enhanced chemiluminescence (Pierce). Autoradiographic signals were quantified using NIH Image. Experiments were repeated in triplicate.

Griess Assay.

Nitric oxide synthase (NOS) activity was measured using the Griess assay to measure nitrite production (Promega, Madison, Wis.). BV-2 cells were plated at 5×10⁴ cells/well in 24-well plates, allowed to reach 90% confluence, and incubated +/−LPS (10 ng/ml)+/−AE1-329 (1 nM-1 μM) or vehicle for 24 h. 501.11 cell culture medium and nitrite standards (0 to 100 nM) were transferred to a 96-well plate and mixed with 50 μl sulphanilamide solution and 50 μl NED solution. After a 10 min incubation at room temperature, absorbance was read at 530 nm on a SpectraMax M5 plate reader (Molecular Devices, Sunnyvale, Calif.). Experiments were repeated in triplicate.

cAMP Dependent Protein Kinase A (PKA) Activity Assay.

PKA activity was determined using the PKA kinase activity assay kit (Assay designs, Ann Arbor, Mich.). Cells were harvested 3 minutes after stimulation, and ELISA was carried out according to the manufacturer's instructions. Kinase activity was calculated as (sample absorbance—blank absorbance)/μg protein and normalized to the average value of vehicle.

ELISA.

Measurements of phospho-Akt and total Akt were determined using the PathScan phospho-Akt (Thr308) and total Akt1 ELISA kits (Cell Signaling Technology, Danvers, Mass.). BV-2 cells were harvested in cell lysis buffer 1 hr after stimulation (20 mM Tris pH7.5, 150 mM NaCl, 1 mM EDTA, 1 mM EGTA, 1% TritonX-100, 2.5 mM sodium pyrophosphate, 1 mM β-glycerophosphate, 1 mM Na₃VO₄, 1 μg/ml leupeptin), and ELISA was carried out according to the manufacturer's instructions. The ratio of phospho-Akt to total Akt was used for statistical analysis.

Immunocytochemical Quantification of NF-κB-p65 Nuclear Translocation.

BV-2 cells were seeded on poly-L-Lysine coated glass coverslips and were maintained in culture for at least 24 hours before treatment with LPS+/−AE1-329. After one hour of treatment, cells were fixed with 4% paraformaldehyde and processed for immunocytochemistry using established protocols. NF-κB cellular localization was detected using rabbit anti-NF-κB p65 antibody (1:200, Santa Cruz Biotechnology) and Cy3-conjugated donkey anti-rabbit secondary antibody (Jackson ImmunoResearch Laboratories, West Grove, Pa.). Nuclei were visualized using Hoechst 33258 dye (MP Biomedicals, Solon, Ohio). Images were acquired using a Nikon Eclipse E600 microscope (Nikon Instruments, Melville, N.Y.) and a Hamamatsu Orca-ER digital camera (Hamamatsu Photonics, Bridgewater, N.J.). For quantification of nuclear NF-κB p65 levels, images were analyzed using the measurements module of Volocity 4.3.2 image analysis software (Improvision Inc., Waltham, Mass.). To define nuclei, a measurements protocol found all areas of the image where the Hoechst signal was above a defined threshold. Further steps separated touching nuclei into individual objects, excluded objects smaller than 30 μm², and excluded objects touching the edge of the image. The program then reported the average intensity of the NF-κB p65 (Cy3) signal for each nucleus. For data quantification, each data point represented the average NF-κB p65 signal from the nuclei in one field of view (>100 cells).

Measurement of F2-Isoprostanes and F4-Neuroprostanes.

Cd11b:EP4f/f and control Cd11bCre:EP4+/+ cerebral cortices were examined for levels of lipid peroxidation by assaying for F2-isoprostanes (F2-IsoPs), which are free radical-generated isomers of prostaglandin PGF₂ in neuronal and non-neuronal cells, and F4-neuroprostanes (F4-NeuroPs), which are neuron-specific products of docosohexanoic acid oxidation using gas chromatography with negative ion chemical ionization mass spectrometry as described previously (Liang et al., 2005).

Isolation of Adult Microglia from Mouse Brain.

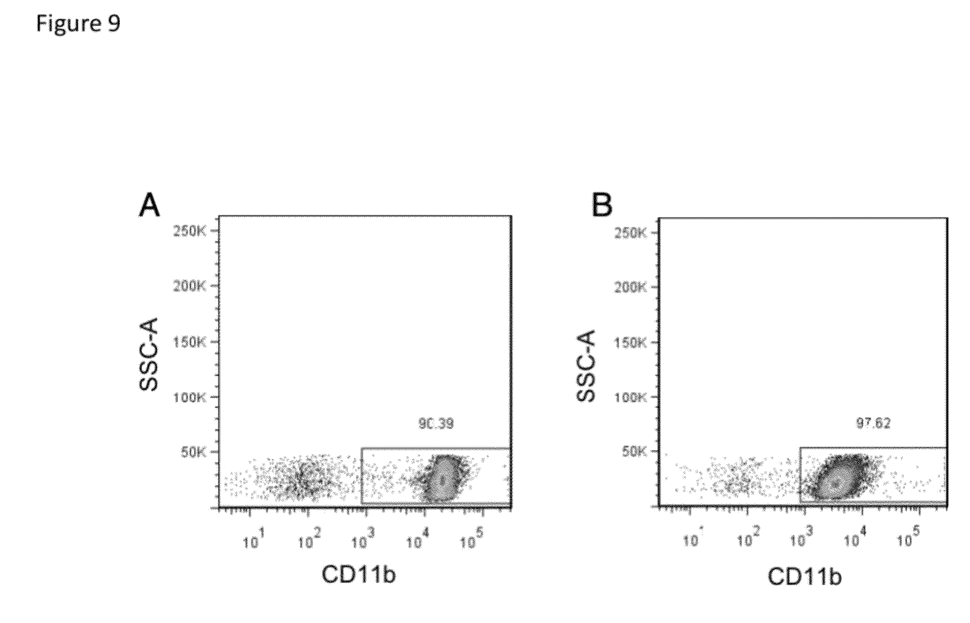

Adult microglial cell isolation was carried according to the methods of Cardona et al., 2006, and cells were processed for RNA isolation or flow cytometry. Mice were deeply anesthetized and perfused with 30 ml ice cold 0.9% saline, and brains were harvested and washed in ice-cold PBS, and individually homogenized using a dounce tissue homogenizer in 4 ml digestion cocktail (RPMI 1640 with 300 U/ml collagenase) and incubated for 45 min at 37° C. Collagenase activity was stopped with the addition of 20 ml of HBSS with 2% fetal bovine serum and 2 mM EDTA. The suspension was triturated and passed through a 100 μM cell strainer (BD Falcon, Bedford, Mass.) and centrifuged at 300×g for 10 min at 4° C. The cell pellet was resuspended in 3.3 ml 75% isotonic Percoll (Sigma, St Louis, Mo.), overlayed with 5 ml 25% isotonic Percoll and 3 ml ice-cold PBS, and spun at 800×g for 60 min at 4° C. without brakes. After centrifugation, cells at the interphase between the 75% and 25% Percoll layers were carefully collected and diluted in 10 ml PBS with 0.5% FBS and 2 mM EDTA and centrifuged at 300×g for 10 min at 4° C. Yields of ˜2.0×10⁵ microglial cells per brain were obtained, consistent with published studies (de Haas et al., 2007), yielding ˜200 ng of microglial RNA per brain. Purity of the microglial preparation was determined in separate experiments by labeling ˜10⁵ cells/ml with phycoerythrin (PE)-conjugated hamster anti-mouse CD11b or IgG isotype control (1:100; eBioscience, San Diego, Calif.) for 30 minutes on ice. Cells were then washed with PBS and fixed (BD, Biosciences, San Diego, Calif.). Flow cytometry was performed on a LSR II (BD Biosciences), and data analyzed with FlowJo 7.2.2 software. Cells obtained by density gradient centrifugation were 90.39% Cd11b positive (FIG. 9A). In separate experiments, magnetic beads conjugated to anti-mouse Cd11b antibody (Miltenyi Biotec, Bergisch Gladbach, Germany) were used to further purify Cd11b positive microglia as described in de Haas et al., 2007. Cells at the Percoll interphase were resuspended in 90 μl ice-cold bead buffer (PBS with 0.5% FBS and 2 mM EDTA, pH7.2) and incubated with 10 μl anti-mouse CD11b-coated beads at 4° C. for 15 min and then rinsed in bead buffer. Cells were pelleted at 300×g for 10 min at 4° C. and separated using a magnetic MACS Cell Separation column (Miltenyi Biotec). Flow cytometry analysis demonstrated that microglia were 97.6% Cd11b positive following this step, however, cell yield was substantially decreased (FIG. 9B). Therefore, cells purified by density centrifugation were used for RNA preparation.

Plasma Multi-Analyte Analysis.

Plasma was analyzed using the Rodent MAP™ Antigens, Version 2.0 multi-analyte profile (Rules Based Medicine, Austin, Tex.) that screens a total of 59 blood secreted proteins using multiplex fluorescent immunoassay.

Statistical Analysis.

Data are presented as mean±standard error of the mean and analyzed using analysis of variance or Student's t test. Prism software (GraphPad Software, Inc. San Diego, Calif.) was used for statistical analyses. Data for Griess assays and quantitative Western analyses were analyzed using one-way or two-way ANOVA, followed by Newman-Keuls multiple comparison or Bonferroni posttest analysis, respectively. For plasma multi-analyte analysis, the concentrations of the 15 plasma proteins that reached statistical significance between LPS+vehicle versus LPS+AE1-329 cohorts were transformed to relative concentrations (Median Z-score). Cluster analysis (Gene Cluster3.0, University of Tokyo, Tokyo and Java TreeView 1.0.13, Alok Saldanda, Calif.) produced a separation of samples according to treatment group and protein levels in plasma. For all data, a probability level of p<0.05 was considered to be statistically significant.

### EXAMPLES

The following examples are put forth so as to provide those of ordinary skill in the art with a complete disclosure and description of how to make and use the present invention; they are not intended to limit the scope of what the inventors regard as their invention. Unless indicated otherwise, part are parts by weight, molecular weight is average molecular weight, temperature is in degrees Centigrade, and pressure is at or near atmospheric.

### Example 1

### Prostaglandin E2 EP4 (PGE2 EP4) Receptor Activation Attenuates Inflammation in BV-2 Cells and Primary Microglia

The immortalised murine microglial cell line BV-2 is often utilized as a valid substitute for primary microglia cells with a normal nitrite oxide production and functional response to IFN-gamma indicating appropriate interaction with T cells and neurons (Henn et al., 2009).

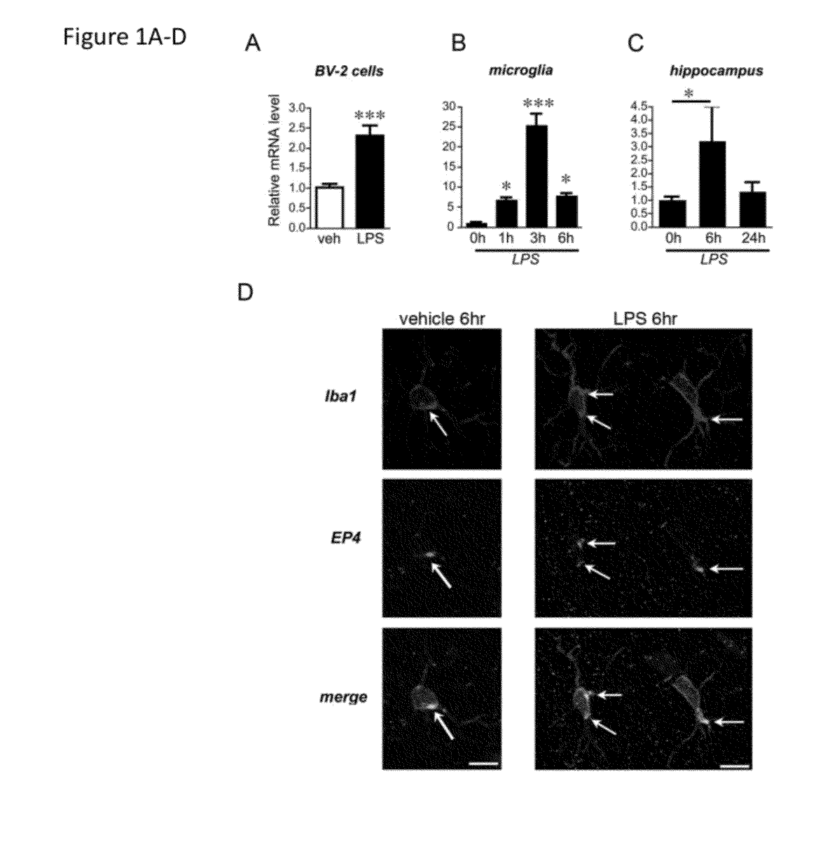

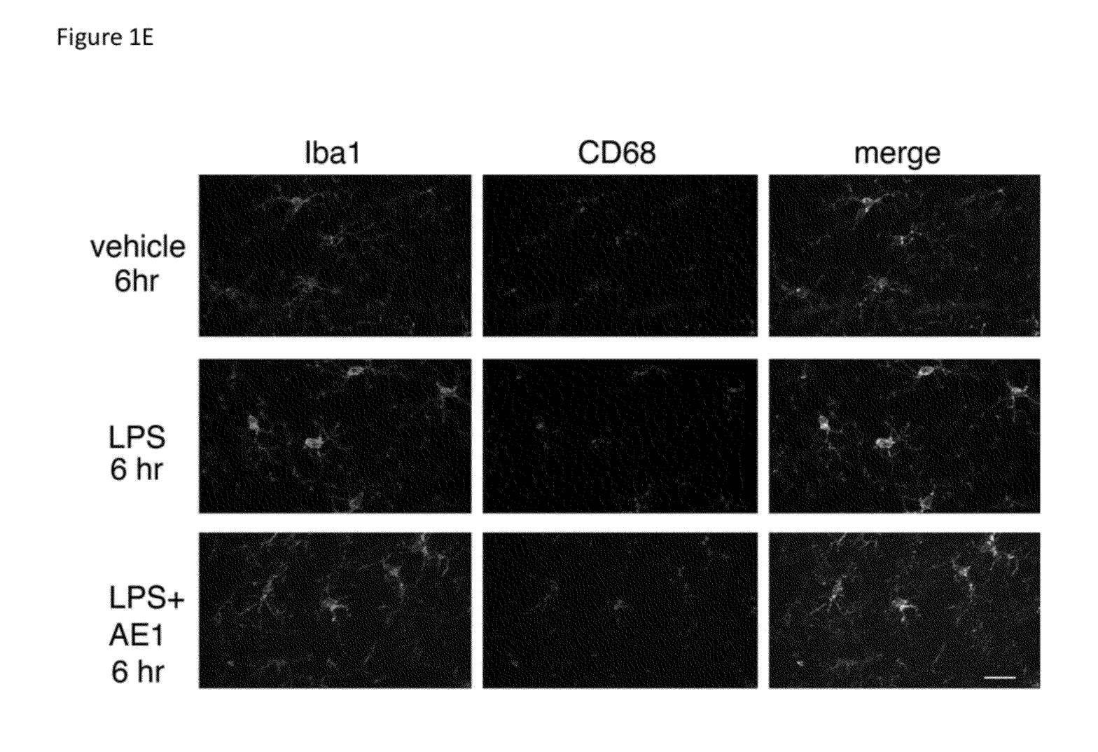

We first determined whether the PGE₂ EP4 receptor is expressed in BV-2 cells and in primary cultured microglia and whether it is regulated in response to lipopolysaccharid (LPS) stimulation. The murine microglial BV-2 cell line exhibits phenotypic and functional properties of microglial cells and has been widely used to model microglial responses (Blasi et al., 1990), in large part because of the limited yields obtained with primary microglial cultures. Quantitative reverse transcriptase polymerase chain reaction (qPCR) demonstrated that EP4 messenger RNA (mRNA) was expressed in both BV-2 and primary cultured microglia (see FIGS. 1A and 1B). Following LPS stimulation (10 ng/ml), there was a rapid increase in EP4 mRNA at 6 hours in BV-2 cells (p<0.01; FIG. 1A). A time course in primary microglia showed a strong induction of mRNA peaking at 3 hours (25.25±2.91 fold increase of relative mRNA expression compared with vehicle-treated cells at 3 hours; ANOVA p<0.0001; FIG. 1B). These data indicate that the EP4 receptor is dynamically upregulated in microglia upon LPS administration. In hippocampus from mice that were stimulated either with LPS or vehicle as control, EP4 mRNA was also dynamically upregulated at 6 hours, returning to baseline by 24 hours (p<0.05; FIG. 1C). Confocal microscopy at 6 hours after stimulation with either vehicle or LPS demonstrated EP4 receptor localization in Iba1 positive microglia in hippocampus in a punctate perinuclear area (arrows, FIG. 1D) in both vehicle and LPS treated mice. Microglia underwent morphological changes by 6 hours after LPS stimulation, as evidenced by induction of cytosolic Iba1 staining and thickening or microglial processes (FIG. 1E). The punctate perinuclear localization of EP4, as well that of other EP receptors to perinuclear areas, has been previously described in other cell types (Bhattacharya et al., 1998 and 1999; Gobeil et al., 2003).

### Example 2

### Prostaglandin E2 EP4 (PGE2 EP4) Receptor Activation Attenuates Lipopolysaccharide-(LPS)-Induced Proinflammatory Gene Expression in BV-2 Cells and Primary Microglia, Involving PKA Activation and Reduction of AKT Phosphorylation

Lipopolysaccharide (LPS, also known as endotoxin) is one of the most powerful microbial stimulants of both innate and specific immune responses; it facilitates the release of inflammatory cytokines, even at far distances from the site of infection or administration, and can even cause shock and death (Beutler, 2000).

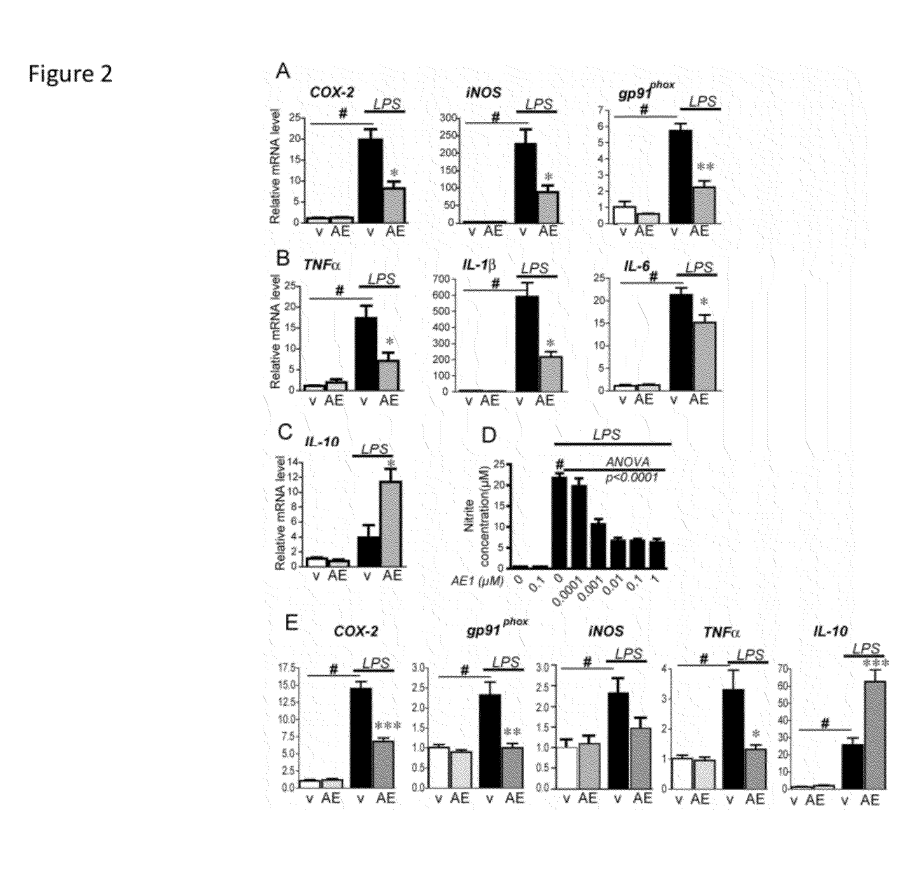

Inflammatory stimuli such as LPS can activate microglia through the CD14/TLR4 receptor complex and induce the expression of pro-inflammatory enzymes and cytokines (Fassbender et al., 2004; Walter et al., 2007). LPS-induced inflammatory responses were tested in the absence and presence of pharmacologic activation of EP4 receptor with the selective EP4 agonist AE1-329. BV-2 cells were treated with LPS (10 ng/ml) in the presence or absence of the selective EP4 agonist AE1-329 (1 μM) for 6 h, and pro-inflammatory gene expression was measured using qPCR (see FIG. 2). LPS significantly induced expression of pro-inflammation enzymes COX-2, iNOS, and the NADPH oxidase subunit gp91^{phox} (#p<0.001); FIG. 2A) as well as canonical pro-inflammatory cytokines including TNF-α, IL-6 and IL-1β (#p<0.001; FIG. 2B). However, co-stimulation with EP4 agonist significantly blunted the induction of these genes (*p<0.05 for COX-2, iNOS and cytokines TNF-α, IL-1β, and IL-6; **p<0.01 for gp91^{phox}). Conversely, co-stimulation with AE1-329 significantly induced expression of the anti-inflammatory IL-10 mRNA (FIG. 2C; *p<0.05). In FIG. 2D, EP4 regulation of iNOS activity was investigated. Nitric oxide (NO) production was significantly elevated at 24 hours in BV-2 cells following LPS treatment (FIG. 2D; #p<0.001). However, co-stimulation with AE1-329 dose-dependently reduced NO production (decreasing 68.9% from 21.76 μM nitrite to 6.57 μM with 10 nM AE1-329; ANOVA p<0.001, post hoc p<0.001 for 1, 10, 100, and 1000 nM AE1-329). Finally, co-stimulation of LPS treated primary microglia with AE1-329 at 3 h also demonstrated a downregulation of pro-inflammatory gene expression (FIG. 2E) and an upregulation of the anti-inflammatory IL-10. Taken together, these data indicate that EP4 activation yields an anti-inflammatory effect in BV-2 cells and primary microglial cells by suppressing the induction of LPS-induced pro-inflammatory gene expression and increasing expression of anti-inflammatory IL-10.

Involvement of PKA Activation and Reduction of Akt Phosphorylation in EP4 Receptor Signaling.

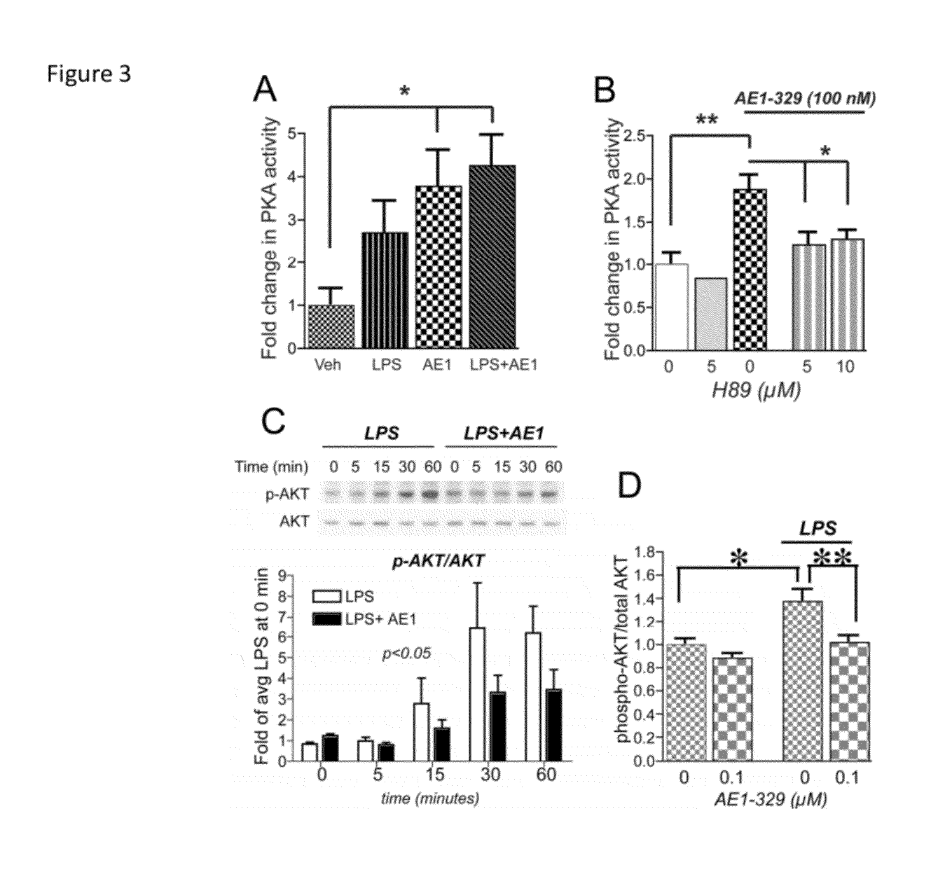

Downstream signaling events for EP4, a Gα-coupled receptor, were investigated in LPS treated BV-2 cells (see FIG. 3). Stimulation of BV-2 cells with AE1-329 or AE1-329 and LPS together significantly increased cAMP dependent protein kinase A (PKA) activity, indicating that the EP4 receptor is positively coupled to cAMP and PKA activation in BV-2 cells. The increase in PKA activity from EP4 receptor signaling was blocked with the PKA inhibitor H89 at doses of 5 μM and 10 μM (FIG. 3B).

In addition to its known Gas coupling to PKA, the EP4 receptor can signal via PI3K and Akt via a Gαi subunit (Fujino et al., 2002; Fujino & Regan, 2006). To further investigate whether EP4 signaling modulates PI3K/Akt pathway activity in LPS-stimulated BV-2 cells, levels of phospho-Akt were measured using quantitative Western analysis (phosphorylated Ser473 Akt; FIG. 3C) and ELISA (phosphorylated Thr308 Akt; FIG. 3D). Phosphorylation at both residues Ser473 and Thr308 is required for Akt activation. Quantitative Western demonstrated a significant attenuation of phospho-Ser473Akt signal in LPS treated BV-2 cells stimulated with EP4 agonist over 60 minutes (p<0.05 for effect of AE1-329 and p<0.001 for effect of time, see FIG. 3 legend). ELISA of phospho-Thr308 Akt also demonstrated a significant decrease in LPS-treated cells at 60 minutes after stimulation with EP4 agonist (p<0.01). Stimulation with EP4 agonist alone did not alter Akt phosphorylation in the absence of LPS. Taken together, these data indicate that EP4 receptor activation in LPS-treated BV-2 cells reduces Akt phosphorylation.

### Example 3

### EP4 Receptor Activation Attenuates LPS—Induced IKK Phosphorylation and Decreases Nuclear Factor-Kappa-B (NF-κB) Nuclear Translocation

We then investigated the anti-inflammatory signaling of EP4 downstream of PI3K/Akt in BV-2 cells. PI3K phosphorylation of Akt can regulate NF-κB nuclear translocation through phosphorylation of the inhibitory I-κB kinase complex (IKK). Phospho-Akt activates the IKK complex by phosphorylating serines on the IKKα and IKKβ subunits (Bai et al., 2009; Barre & Perkins, 2007; Gustin et al., 2001; Ozes et al., 1999; Delhase et al., 2000) and activated IKK phosphorylates I-κB and targets it for degradation, allowing NF-κB to translocate to the nucleus (DiDonato et al., 1997). Nuclear translocation of NF-κB induces expression of many proinflammatory genes including COX-2, iNOS, TNF-α, IL-1β, and IL-6. Because of the broad range of pro-inflammatory genes downregulated by EP4 signaling in microglial cells, we tested whether EP4 affected NF-κB activation and nuclear translocation in LPS-stimulated BV-2 cells.

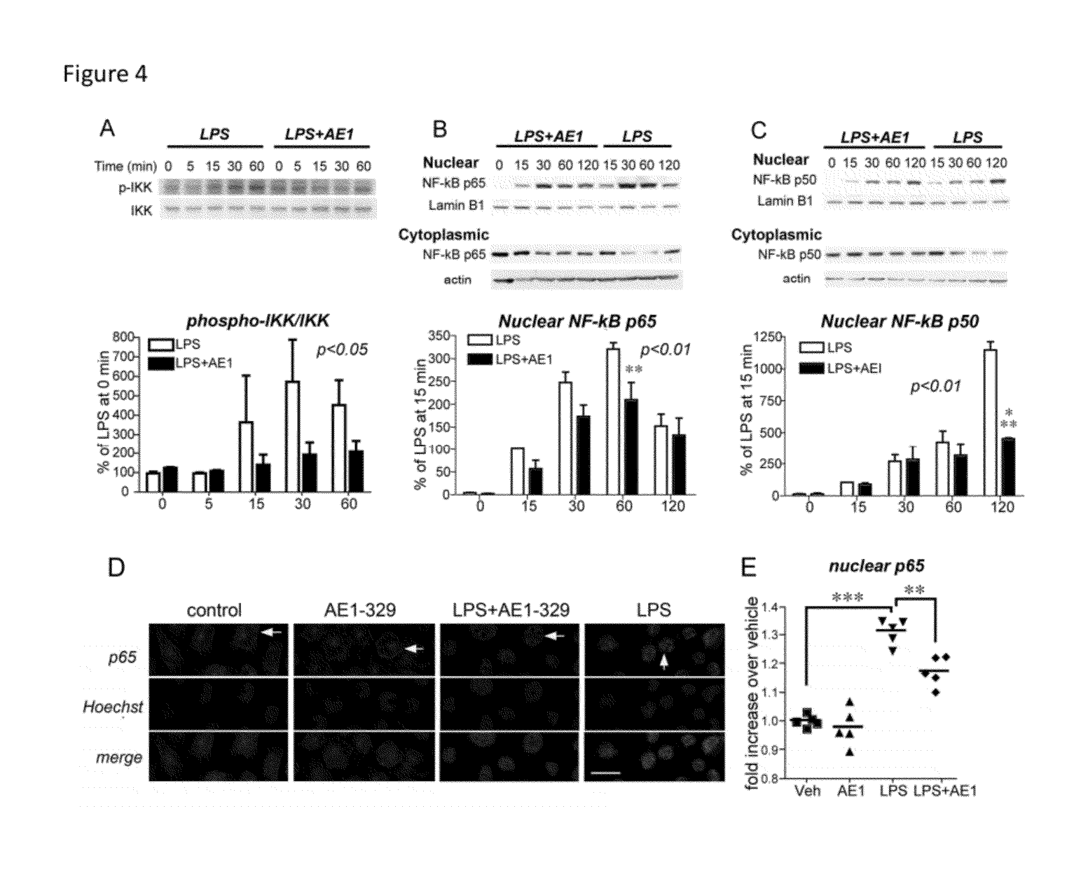

LPS stimulation induced the phosphorylation of IKK, but this was attenuated by co-stimulation with the EP4 agonist AE1-329 (FIG. 4A; p<0.05 2-way ANOVA). In addition, LPS treatment induced a time-dependent nuclear translocation of NF-κB subunits p65 and p50, but co-stimulation with AE1-329 reduced levels of NF-κB nuclear translocation (FIGS. 4B and 4C; p<0.01 2-way ANOVA for both p65 and p50) as compared to vehicle; moreover cytoplasmic levels of p65 and p50 were increased in LPS-treated cells stimulated with AE1-329 as compared to vehicle stimulated cells. Semi-quantitative measurements of p65 immunofluorescent signal also revealed an increase in nuclear translocation with LPS treatment (FIGS. 4D and E; p<0.001) that was significantly attenuated with co-stimulation of EP4 receptor (p<0.01). Therefore, EP4 receptor activation decreased LPS-induced phosphorylation of Akt and IKK, and decreased translocation of NF-κB subunits p65 and p50 to the nucleus, providing a potential mechanism for its downregulation of pro-inflammatory genes.

### Example 4

### CD11BCRE-Mediated Conditional Deletion of EP4 Receptor Leads to Increased LPS—Induced Pro-Inflammatory Gene Expression and Lipid Peroxidation in Brain

Cd11bCre:EP4+/+ and Cd11bCre:EP4f/f mice were generated, in which the EP4 receptor is selectively deleted in cells of monocytic lineage, including microglia and macrophages, to investigate whether conditional deletion of EP4 in microglia/macrophages would lead to increased pro-inflammatory gene expression following LPS administration. Increased pro-inflammatory protein expression and activity would likely lead to increased inflammatory oxidative stress, leading to increases in lipid peroxidation which could be reliably measured by 24 hours after LPS stimulation (Liang et al., 2005 and 2008). Therefore, a time point of 24 hours after peripheral administration of LPS to the Cd11bCre:EP4+/+ and Cd11bCre:EP4f/f mice was chosen to test for increased pro-inflammatory gene expression following LPS administration.

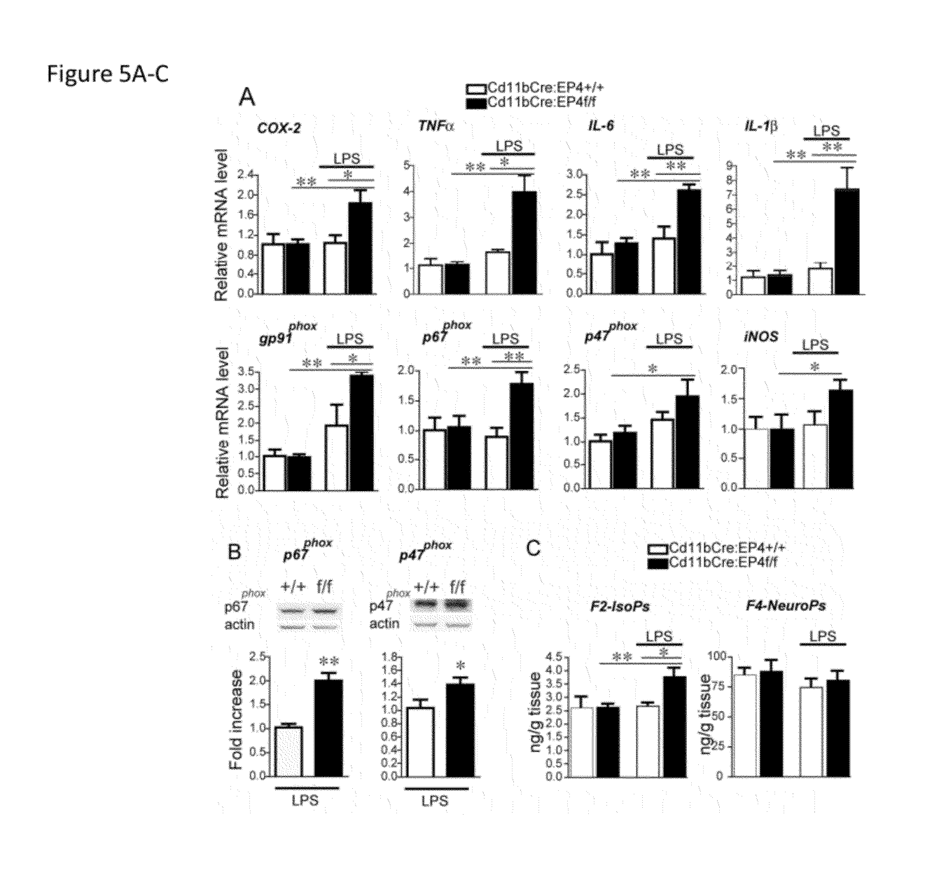

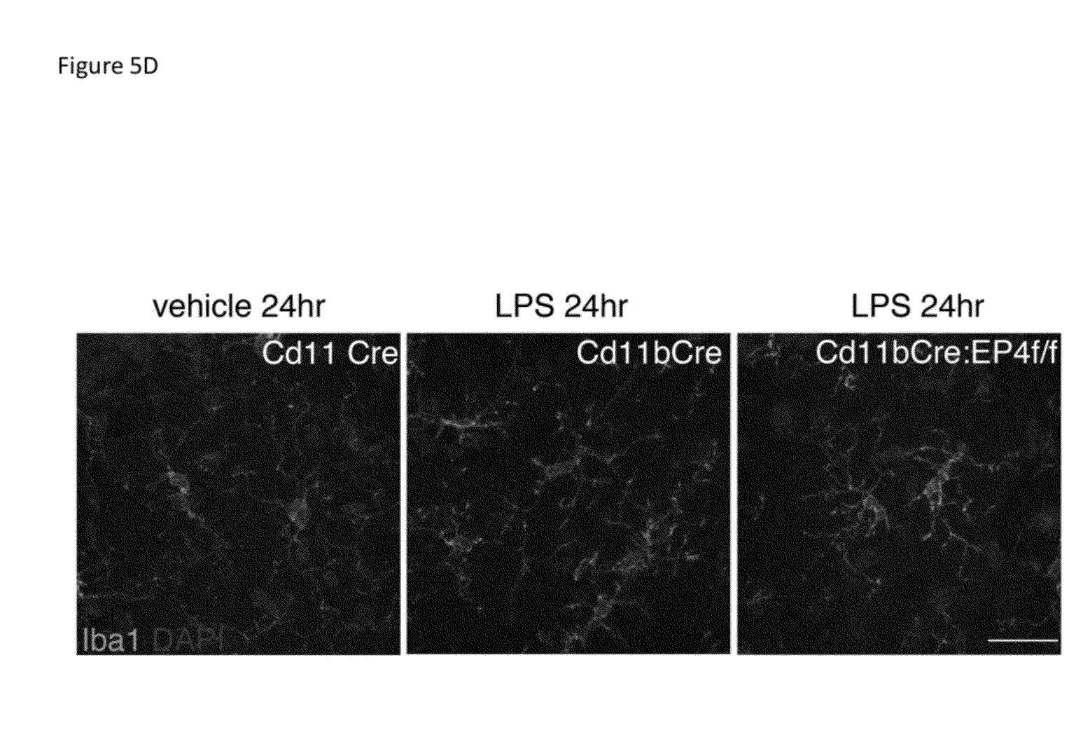

Peripheral stimulation with LPS results in peripheral and CNS inflammatory responses (Lund et al., 2006; Laye at al., 1994). Because this neuroinflammatory response can induce synaptic and neuronal injury and disrupt hippocampal-dependent memory, we examined pro-inflammatory gene expression in hippocampus in Cd11bCre:EP4f/f mice and control Cd11bCre:EP4+/+ littermates treated with LPS (5 mg/kg i.p.; FIG. 5). At 24 hours after LPS stimulation, there was no difference in pro-inflammatory gene expression between vehicle and LPS-treated wild-type mice, reflecting the documented resolution of the inflammatory response by that time point (Lund et al., 2006). However, in EP4 conditional knockout mice stimulated with LPS, expression of COX-2, TNF-α, IL-6, IL-1β as well as subunits of the NADPH oxidase complex, including gp91^{phox}, p67^{phox} and p47^{phox} were all significantly upregulated at 24 hours (FIGS. 5A, B). Levels of cerebral cortical lipid peroxidation showed no differences in levels of neuronal-specific F4 neuroprostanes, however levels of F2-isoprostanes were significantly higher in Cd11b:EP4f/f cerebral cortices compared with Cd11b: EP4+/+ mice (p<0.05). Arachidonic acid, a major component of membrane phospholipids in all brain cell types, is particularly vulnerable to free radical attack and its peroxidation is reflected in the F2-isoprostane measurements. These in vivo data complement the in vitro findings in cultured microglial cells, and indicate that EP4 functions in an anti-inflammatory manner in vivo in brain inflammation. However, in spite of the increased pro-inflammatory gene expression and lipid peroxidation, we did not observe overt differences in hippocampal microglial morphology between genotypes following LPS administration at 24 hours (FIG. 5D).

### Example 5

### Effect of EP4 Agonist on LPS-Induced Innate Immunity In Vivo

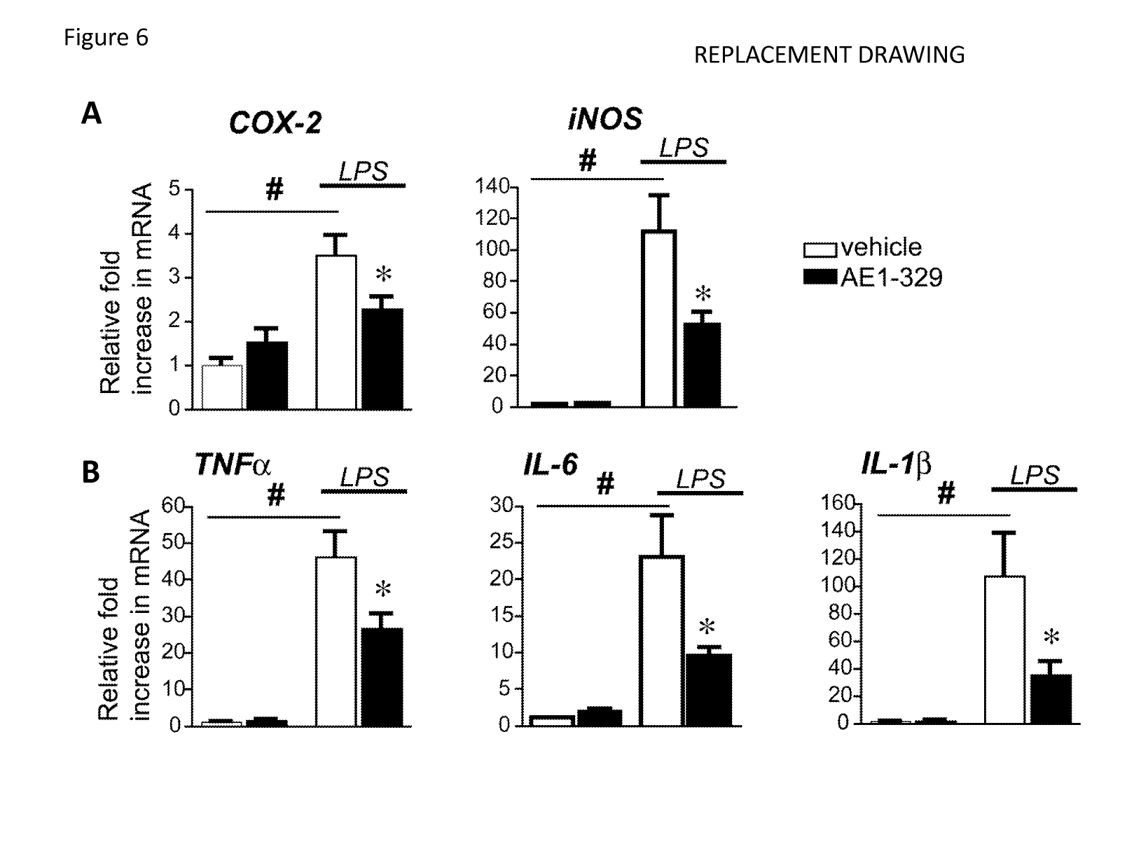

In vitro stimulation of LPS-treated microglial BV-2 cells and primary microglia resulted in a broad downregulation of pro-inflammatory gene expression by 6 hours after stimulation (see Example 2, FIG. 2). To confirm a similar acute anti-inflammatory effect of EP4 signaling in vivo, we treated mice with LPS (5 mg/kg, i.p.) with or without EP4 agonist AE1-329 (300 μg/kg, s.c.; FIG. 6) and examined mRNA expression at 6 hours, a similar time point to that used in vitro. LPS led to significant increases in hippocampal mRNA of pro-inflammatory cytokines TNF-α, IL-6, IL-1β as well as COX-2, iNOS, and the NADPH oxidase subunits gp91^{phox}, p67^{phox}, and p47^{phox} genes (not shown) at 6 hours after LPS. Co-administration of EP4 agonist significantly attenuated LPS-induced COX-2, iNOS, TNF-α, IL-6, and IL-1β mRNA levels in hippocampus; there was a trend toward decreased expression of NADPH oxidase subunit gp91^{phox}, p67^{phox}, and p47^{phox} (not shown). Thus, peripheral administration of a selective EP4 agonist significantly blunted the CNS inflammatory response to systemic LPS in a time course similar to in vitro studies in BV-2 cells and primary microglia. Microglial morphological changes in response to LPS appeared modestly decreased with co-administration of EP4 agonist (FIG. 1E).

### Example 6

### EP4 Signaling Regulates Inflammatory Gene Expression in Microglia Isolated from Adult Brain

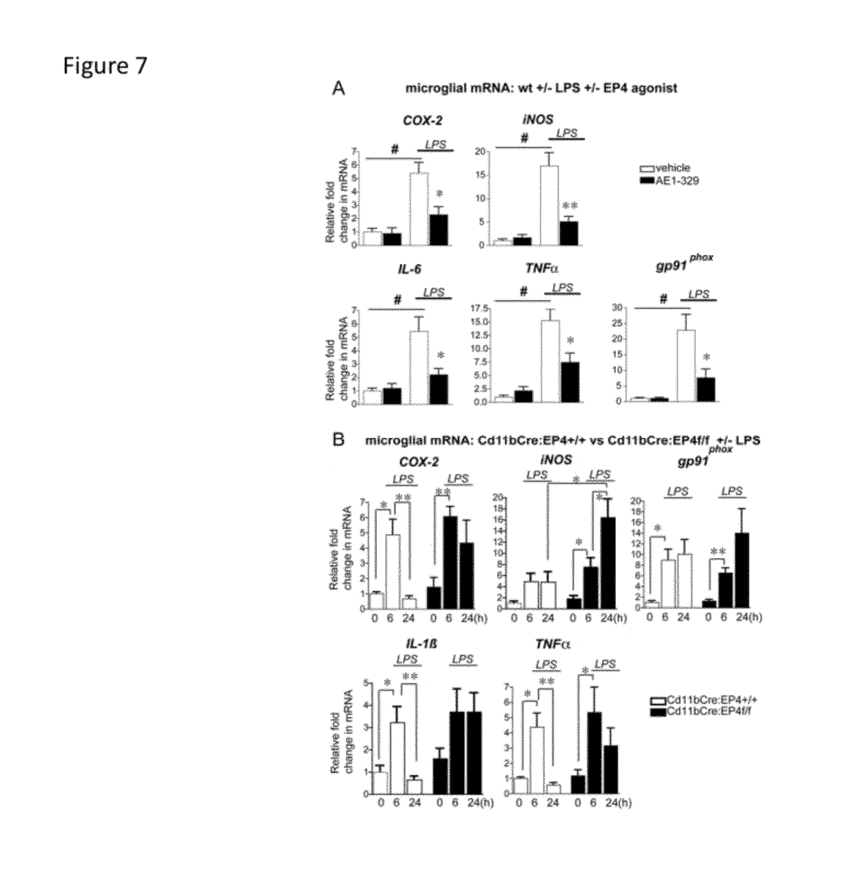

To further confirm that EP4 signaling regulated expression of inflammatory genes in brain microglia in vivo, microglia were acutely isolated from wild type adult mice stimulated +/−LPS+/−EP4 agonist (FIG. 7A) and from Cd11bCre:EP4f/f and Cd11bCre:EP4+/+ mice stimulated +/−LPS (FIG. 7B). For pharmacological experiments (FIG. 7A), 2-3 mo C57B6 male mice (n=6-8 per group) received an injection of saline or LPS (5 mg/kg, i.p.)+/−vehicle or AE1-329 (30014/kg subcutaneously) and microglia were harvested for RNA isolation at 6 hours, similar to the time point used for post-natal microglia (see FIG. 2E). Administration of LPS led to significant increases in microglial COX-2, iNOS, IL-6, TNF-α, and gp91^{phox} that were significantly reduced with co-administration of EP4 agonist. Conversely, genetic experiments examining microglia isolated from adult Cd11bCre:EP4+/+ and Cd11bCre:EP4f/f C57B6 mice+/−LPS demonstrated that the normal downregulation of pro-inflammatory gene expression at 24 hours in Cd11bCre:EP4+/+ mice was blocked in the Cd11bCre:EP4f/f mice. In this experiment, we also tested a 6 hour time point, which did not show a difference in inflammatory gene induction between genotypes. Thus, microglial EP4 deletion results in persistently elevated inflammatory gene expression at 24 hours, confirming similar findings in whole hippocampal mRNA (see FIG. 5).

### Example 7

### EP4 Receptor Activation Attenuates Plasma Cytokine Levels Induced in Response to LPS

The innate immune response in brain to systemic inflammation can occur as a direct response in brain or as a peripheral-to-central immune response, in which serum cytokines either are transported across the blood-brain barrier or act on endothelium to transduce the inflammatory response to brain parenchyma. IL-6, IL-1β, and TNF-α are generated as part of peripheral inflammation by macrophages, and are well-documented effectors of peripheral-to-central immune responses where peripheral inflammatory signals lead to expression of cytokines in brain. In the case of the EP4 conditional knockout (cKO), where EP4 is deleted in peripheral macrophages as well as CNS microglia, the anti-inflammatory effects of EP4 may be mediated by brain microglia, peripheral macrophages, or both. Moreover, the effects of peripherally administered AE1-329 on hippocampal inflammation could be due to anti-inflammatory effects of microglial EP4, macrophage EP4, or both.

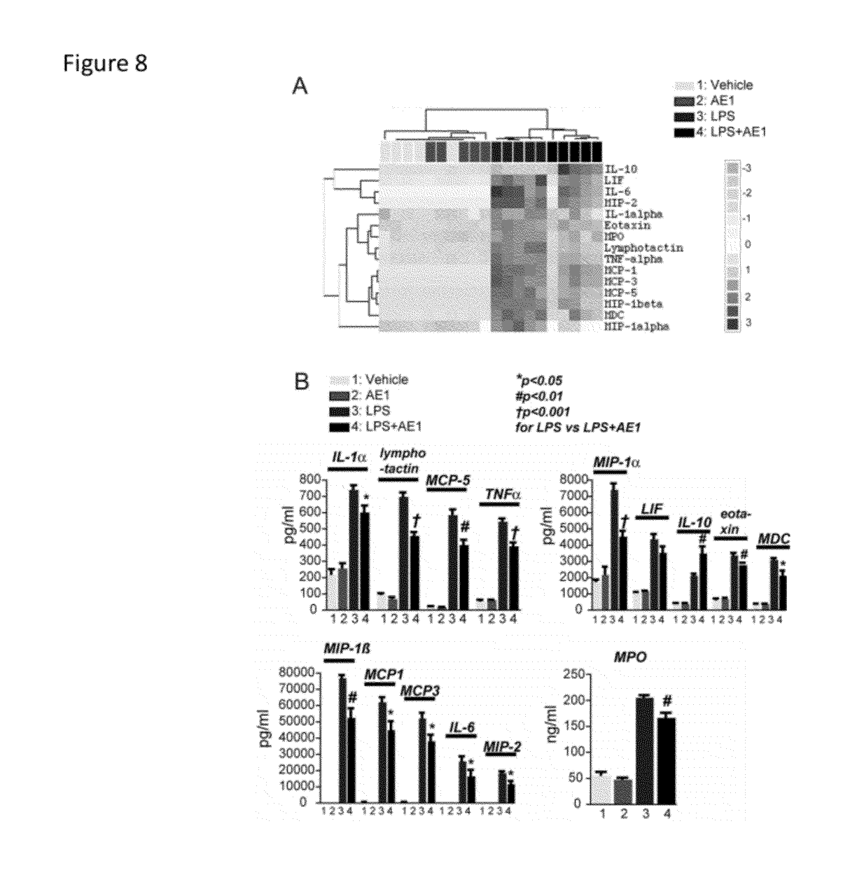

To address specifically whether peripheral EP4 signaling could modulate central inflammatory processes, we used a proteomic approach and examined plasma secreted proteins from mice stimulated with LPS+/−AE1-329. Previous studies have demonstrated a very rapid induction of pro-inflammatory cytokines in response to LPS within 2-4 hours (Lund et al., 2006; Rosenberger et al., 2000) so we selected an early time point of 3 hours after LPS administration to test the effects of selective activation of peripheral EP4 signaling. Co-administration of AE1-329 had a significant and broad anti-inflammatory effect on plasma cytokine and chemokine levels in LPS stimulated mice (FIGS. 8A and B). EP4 receptor activation in LPS-treated mice significantly decreased levels of cytokines TNF-α, IL-1α, eotaxin, and chemokines MDC, MIP-1α, MIP-1β, MIP-1γ, MIP-2, MCP-1, MCP-3, and MCP-5, and reduced secreted levels of myeloperoxidase; levels of IL-6 and LIF showed a trend towards decreased levels at 6 h. Finally, the administration of the AE1-329 EP4 agonist significantly increased plasma levels of the anti-inflammatory IL-10. Thus, peripherally administered EP4 agonist blunted serum (FIG. 8) as well as hippocampal inflammatory responses (FIG. 6). This suggests that peripheral-to-central innate immune responses may be modulated in a beneficial manner by selectively targeting the EP4 receptor.

### Example 8

### EP4 Receptor Activation Attenuates Lipopolysaccharide (LPS)-Induced Release of Proinflammatory Cytokines in Primary Human Monocytes

Primary human monocytes were stimulated with lipopolysaccharide (LPS) in the absence and presence of the EP4 agonist AE1-329, and the resulting levels of TNF-α and IL-6 were investigated. IL-6 and TNF-α are two canonical inflammatory cytokines that are generated by macrophages in the course of peripheral inflammation, and are well-documented effectors of peripheral-to-central immune responses where peripheral inflammatory signals lead to expression of cytokines in the brain.

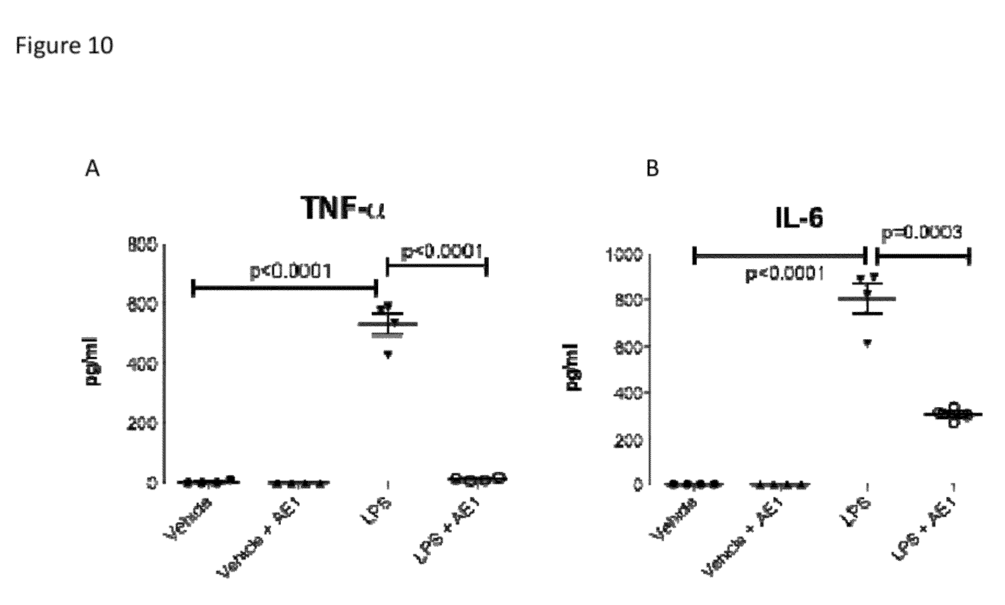

As shown in FIG. 10, human EP4 receptor activation in LPS-stimulated human monocytes had a distinct anti-inflammatory effect and significantly decreased levels of IL-6 and TNF-α. Primary human monocytes were stimulated with either vehicle (PBS) alone, AE1-329 (100 nM), LPS (100 ng/ml), and LPS (100 ng/ml) plus AE1-329 (100 nM). Two dilutions (1:5 and 1:10) were tested using anti-human IL6 and anti-human TNF-α ELISA to measure concentrations of these two cytokines in pg/ml after 6 hours of stimulation. As illustrated in panel A of FIG. 10, 6 hours after stimulation with LPS a significant increase in TNF-α levels was observed which was entirely reversed when the EP4 agonist AE1-329 was co-administered. As shown in panel B of FIG. 10, 6 hours after stimulation with LPS a significant increase in IL-6 levels was noticeable, which was significantly reduced when the EP4 agonist AE1-329 was co-administered.

Although the foregoing invention and its embodiments have been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope.

### REFERENCES

Abreu M T & Arditi M (2004). Innate immunity and toll-like receptors: clinical implications of basic science research. J Pediatr 144:421-429.
Bai D et al. (2009). Akt-mediated regulation of NFkappaB and the essentialness of NFkappaB for the oncogenicity of PI3K and Akt. Int J Cancer 125:2863-2870.
Barre B & Perkins N D (2007). A cell cycle regulatory network controlling NF-kappaB subunit activity and function. Embo J 26:4841-4855.
Beutler B (2000). T1r4: central component of the sole mammalian LPS sensor. Curr Opin Immunol 12:20-26.
Bhattacharya M et al. (1998). Nuclear localization of prostaglandin E2 receptors. Proc Natl Acad Sci USA 95:15792-15797.
Bhattacharya M et al. (1999). Localization of functional prostaglandin E2 receptors EP3 and EP4 in the nuclear envelope. J Biol Chem 274:15719-15724.
Blasi E et al. (1990). Immortalization of murine microglial cells by a v-raf/v-myc carrying retrovirus. Journal of neuroimmunology 27:229-237.
Boillee S et al. (2006). Onset and progression in inherited ALS determined by motor neurons and microglia. Science 312:1389-1392.
Cardona A E et al. (2006). Isolation of murine microglial cells for RNA analysis or flow cytometry. Nature protocols 1:1947-1951.
Coleman R A et al. (1994). International Union of Pharmacology classification of prostanoid receptors: properties, distribution, and structure of the receptors and their subtypes. Pharmacol Rev 46:205-229.
de Haas A H et al. (2007). Optimized isolation enables ex vivo analysis of microglia from various central nervous system regions. Glia 55:1374-1384.
Delhase M et al. (2000). Kinase regulation in inflammatory response. Nature 406:367-368.
DiDonato J A et al. (1997). A cytokine-responsive IkappaB kinase that activates the transcription factor NF-kappaB. Nature 388:548-554.
Fassbender K et al. (2004). The LPS receptor (CD14) links innate immunity with Alzheimer's disease. Faseb J 18:203-205.
Fujino H et al. (2002). Phosphorylation of glycogen synthase kinase-3 and stimulation of T-cell factor signaling following activation of EP2 and EP4 prostanoid receptors by prostaglandin E2. J Biol Chem 277:2614-2619.
Fujino H & Regan J W (2006). EP(4) prostanoid receptor coupling to a pertussis toxin-sensitive inhibitory G protein. Mol Pharmacol 69:5-10.
Gobeil F et al. (2003). Nuclear prostaglandin signaling system: biogenesis and actions via heptahelical receptors. Can J Physiol Pharmacol 81:196-204.
Gustin J A et al. (2001). The PTEN tumor suppressor protein inhibits tumor necrosis factor-induced nuclear factor kappa B activity. J Biol Chem 276:27740-27744.
Hayashi R et al. (2011). Efficient synthesis of a novel m-phenylene derivative as a selective EP4 agonist inducing follicular growth and maturation in the ovary. Bioorg & Med Chem Letters 21(24):7505-7508.
Henn A et al. (2009). The suitability of BV2 cells as alternative model system for primary microglia cultures or for animal experiments examining brain inflammation. ALTEX 26:83-94.
Jiang G-L et al. (2007). The Prevention of Colitis by E Prostanoid Receptor δ Agonist through Enhancement of Epithelium Survival and Regeneration. J Pharm Exp Ther 320:22-28.
Laye S et al. (1994). Peripheral administration of lipopolysaccharide induces the expression of cytokine transcripts in the brain and pituitary of mice. Brain Res Mol Brain Res 27:157-162.
Letiembre M et al. (2007a). Screening of innate immune receptors in neurodegenerative diseases: A similar pattern. Neurobiol Aging 30:759-768.
Letiembre M et al. (2007b). Innate immune receptor expression in normal brain aging. Neuroscience 146:248-254.
Leung L & Cahill CM (2010). TNF-α and neuropathic pain—a review. J Neuroinflamm 7:27-37.
Liang X et al. (2005). Deletion of the prostaglandin E2 EP2 receptor reduces oxidative damage and amyloid burden in a model of Alzheimer's disease. J Neurosci 25:10180-10187.
Liang X et al. (2008). The prostaglandin E2 EP2 receptor accelerates disease progression and inflammation in a model of amyotrophic lateral sclerosis. Ann Neurol 64:304-314.
Liu Y et al. (2008). Endotoxin induces a delayed loss of TH-IR neurons in substantia nigra and motor behavioral deficits. Neurotoxicology 29:864-870.
Lull M E & Block ML (2010). Microglial activation and chronic neurodegeneration. Neurotherapeutics 7: 354-365.
Lund S et al. (2006). The dynamics of the LPS triggered inflammatory response of murine microglia under different culture and in vivo conditions. J Neuroimmunol 180:71-87.
McDermott A M et al. (2006). The burden of neuropathic pain: results from a cross-sectional survey. Eur J Pain 10:127-135.
McGeer P L & McGeer E G (2004). Inflammation and the degenerative diseases of aging. Ann N Y Acad Sci 1035:104-116.
Nagamatsu T et al. (2006). Protective effect of prostaglandin EP4-receptor agonist on anti-glomerular basement membrane antibody-associated nephritis. J Pharmacol Sci 102:182-188.
Narumiya S et al. (1999). Prostanoid Receptors: Structures, Properties, and Functions. Physiol. Rev. 79, pp. 1193-1226.
Nguyen M D et al. (2002). Innate immunity: the missing link in neuroprotection and neurodegeneration? Nature reviews 3:216-227.
Ozes O N et al. (1999). NF-kappaB activation by tumour necrosis factor requires the Akt serine-threonine kinase. Nature 401:82-85.
Qin L et al. (2007). Systemic LPS causes chronic neuroinflammation and progressive neurodegeneration. Glia 55:453-462.
Rosenberger C M et al. (2000). Salmonella typhimurium infection and lipopolysaccharide stimulation induce similar changes in macrophage gene expression. J Immunol 164:5894-5904.
Schneider A et al. (2004). Generation of a conditional allele of the mouse prostaglandin EP4 receptor. Genesis 40:7-14.
Shibuya I et al. (2002). Involvement of postsynaptic EP4 and presynaptic EP3 receptors in actions of prostaglandin E2 in rat supraoptic neurones. Journal of Neuroendocrinology 14:64-72.
Suzawa T C et al. (2000). The role of prostaglandin E receptor subtypes (EP1, EP2, EP3, and EP4) in bone resorption: an analysis using specific agonists for the respective EPs. Endocrinology 141:1554-1559.
Walter S et al. (2007). Role of the toll-like receptor 4 in neuroinflammation in Alzheimer's disease. Cell Physiol Biochem 20:947-956.
Yoshida K et al. (2002). Stimulation of bone formation and prevention of bone loss by prostaglandin EP4 receptor activation. PNAS 99(7):4580-4585.
Zhang J & Rivest S (1999). Distribution, regulation and colocalization of the genes encoding the EP2- and EP4-PGE2 receptors in the rat brain and neuronal responses to systemic inflammation. Eur J Neurosci 11:2651-2668.

What is claimed is:

1. A method of attenuating neuronal inflammation and neuronal damage in a human subject following acute central nervous system injury, the method comprising administering a pharmaceutical composition comprising a prostaglandin E2 EP4 receptor agonist to said human subject in a dosage and dosing regimen effective to attenuate neuronal inflammation and neuronal damage.

2. The method of claim 1, wherein said acute central nervous system injury is caused by a traumatic brain injury, cerebral ischemia, cerebral glucose deprivation, cerebral oxidative stress, spinal cord injury or excitotoxic injury.

3. The method of claim 1, wherein said prostaglandin E2 EP4 receptor agonist is AE1-329.

4. The method of claim 1, wherein said prostaglandin E2 EP4 receptor agonist is an analog of AE1-329.

5. The method of claim 1, wherein said prostaglandin E2 EP4 receptor agonist is a prodrug of AE1-329 or a prodrug of an AE1-329 analog.

6. A method of attenuating neuronal inflammation and neuronal damage in a human subject suffering from a chronic central nervous system injury, the method comprising administering a pharmaceutical composition comprising a prostaglandin E2 EP4 receptor agonist to said human subject in a dosage and dosing regimen effective to attenuate neuronal inflammation and neuronal damage.

7. The method of claim 6, wherein said chronic central nervous system injury is caused by a neurodegenerative disease.

8. The method of claim 7, wherein said neurodegenerative disease is Alzheimer's disease, Parkinson's disease, Amyotrophic Lateral Sclerosis or Huntington's disease.

9. The method of claim 6, wherein said prostaglandin E2 EP4 receptor agonist is AE1-329.

10. The method of claim 6, wherein said prostaglandin E2 EP4 receptor agonist is an analog of AE1-329.

11. The method of claim 6, wherein said prostaglandin E2 EP4 receptor agonist is a prodrug of AE1-329 or a prodrug of an AE1-329 analog.

12. A method of attenuating neuronal inflammation and neuronal damage in a human subject at risk of developing a chronic central nervous system injury, the method comprising administering a pharmaceutical composition comprising a prostaglandin E2 EP4 receptor agonist to said human subject prior to onset of symptoms of a chronic central nervous system injury in a dosage and dosing regimen effective to attenuate neuronal inflammation and neuronal damage.

13. The method of claim 12, wherein said chronic central nervous system injury is caused by a neurodegenerative disease.

14. The method of claim 13, wherein said neurodegenerative disease is Alzheimer's disease, Parkinson's disease, Amyotrophic Lateral Sclerosis or Huntington's disease.

15. The method of claim 12, wherein said prostaglandin E2 EP4 receptor agonist is AE1-329.

16. The method of claim 12, wherein said prostaglandin E2 EP4 receptor agonist is an analog of AE1-329.

17. The method of claim 12, wherein said prostaglandin E2 EP4 receptor agonist is a prodrug of AE1-329 or a prodrug of an AE1-329 analog.

18. A method of treating neuropathic pain caused by an inflammatory response in a human subject, the method comprising administering a pharmaceutical composition comprising a prostaglandin E2 EP4 receptor agonist to said human subject in a dosage and dosing regimen effective to treat neuropathic pain, whereby said agonist reduces said inflammatory response by reducing levels of one or more inflammatory cytokines.

19. The method of claim 18, wherein said prostaglandin E2 EP4 receptor agonist is AE1-329.

20. The method of claim 18, wherein said prostaglandin E2 EP4 receptor agonist is an analog of AE1-329.

21. The method of claim 18, wherein said prostaglandin E2 EP4 receptor agonist is a prodrug of AE1-329 or a prodrug of an AE1-329 analog.

22. The method of claim 18, wherein the cytokine is selected from the group consisting of tumor necrosis factor-alpha, IL1β and IL-6.


SEQUENCE LISTING (22 sequences)

SEQ ID NO: 1 - DNA, 20 bp, Artificial Sequence
  Primer
cggctaccacatccaaggaa

SEQ ID NO: 2 - DNA, 18 bp, Artificial Sequence
  Primer
gctggaattaccgcggct

SEQ ID NO: 3 - DNA, 24 bp, Artificial Sequence
  Primer
agacaccacctcgctgagaacttt

SEQ ID NO: 4 - DNA, 24 bp, Artificial Sequence
  Primer
aacctcatccaccaacaggacact

SEQ ID NO: 5 - DNA, 21 bp, Artificial Sequence
  Primer
gccggagacgccagaagagct

SEQ ID NO: 6 - DNA, 21 bp, Artificial Sequence
  Primer
ggggctgcgactgagggtgaa

SEQ ID NO: 7 - DNA, 21 bp, Artificial Sequence
  Primer
ccaactgggataacgagttca

SEQ ID NO: 8 - DNA, 21 bp, Artificial Sequence
  Primer
gagagtttcagccaaggcttc

SEQ ID NO: 9 - DNA, 24 bp, Artificial Sequence
  Primer
tacagcaaaggacaggactgggtt

SEQ ID NO: 10 - DNA, 24 bp, Artificial Sequence
  Primer
gaggcacttggctttctgcaaact

SEQ ID NO: 11 - DNA, 21 bp, Artificial Sequence
  Primer
tgacggcaaacatgacttcag

SEQ ID NO: 12 - DNA, 18 bp, Artificial Sequence
  Primer
gccatcgggcatctggta

SEQ ID NO: 13 - DNA, 20 bp, Artificial Sequence
  Primer
tgcaagatccacagcctacc

SEQ ID NO: 14 - DNA, 20 bp, Artificial Sequence
  Primer
gctcagttgaacgccttttg

SEQ ID NO: 15 - DNA, 24 bp, Artificial Sequence
  Primer
gggttgccaagccttatcggaaat

SEQ ID NO: 16 - DNA, 24 bp, Artificial Sequence
  Primer
tcttcagcttctcacccagggaat

SEQ ID NO: 17 - DNA, 24 bp, Artificial Sequence
  Primer
gatctcaaagacaaccaacatgtg

SEQ ID NO: 18 - DNA, 24 bp, Artificial Sequence
  Primer
ctccagctggaagactcctcccag

SEQ ID NO: 19 - DNA, 22 bp, Artificial Sequence
  Primer
ccaggatgaggacatgagcacc

SEQ ID NO: 20 - DNA, 23 bp, Artificial Sequence
  Primer
ttctctgcagactcaaactccac

SEQ ID NO: 21 - DNA, 22 bp, Artificial Sequence
  Primer
catagctacctggagtacatga

SEQ ID NO: 22 - DNA, 22 bp, Artificial Sequence
  Primer
cattcatattgtcagttcttcg